(19) United States
(12) Patent Application Publication
(10) Pub. No.: US 2025/0129340 A1
(43) Pub. Date: Apr. 24, 2025

(54) HYDROGELS FOR ORGANOID CULTURE

(71) Applicants: UNIVERSITEIT MAASTRICHT, Maastricht (NL); ACADEMISCH ZIEKENHUIS MAASTRICHT, Maastricht (NL)

(72) Inventors: Floor Agnes Andrea RUITER, Maastricht (NL); Matthew Brandon BAKER, Maastricht (NL); Vanessa LAPOINTE, Maastricht (NL); Francis L.C. MORGAN, Maastricht (NL)

(21) Appl. No.: 18/706,271

(22) PCT Filed: Nov. 1, 2022
(86) PCT No.: PCT/EP2022/080452

§ 371 Date: Apr. 30, 2024

(30) Foreign Application Priority Data
Nov. 1, 2021 (EP) 21205818.4

(51) Int. Cl. C12N 5/071; C12N 5/00

(52) U.S. Cl. CPC C12N 5/0686; C12N 5/0062; C12N 2501/119; C12N 2506/45; C12N 2513/00; C12N 2533/30; C12N 2533/54; C12N 2537/10

(57) ABSTRACT

The invention relates to a hydrogel from crosslinked polymers with a defined stiffness. The described hydrogels are particularly useful for growing organoids such as for example kidney organoids. The invention further relates to methods of manufacturing the hydrogels and to methods of culturing organoids, as well as uses of the obtained hydrogels.

### FIELD OF THE INVENTION

The invention relates to the field of organoid culturing. The invention particularly relates to methods to manufacture hydrogels with improved properties for culturing organoids such as kidney organoids. The invention further relates to hydrogels obtained by the methods described herein and uses thereof.

### INTRODUCTION

Within the organoid field, various hydrogels have been investigated to influence cell behaviour, including biopolymer-based hydrogels (e.g. alginate), fully synthetic materials (polyethylene glycol (PEG) and polyacrylamide), and bio hybrids (Matrigel combined with PEG, fibrin, or alginate). Existing synthetic hydrogels for organoid culture largely rely on covalent or non-reversible cross-linking interactions, while the natural extracellular matrix (ECM) is dynamic. Dynamic covalent cross-linked hydrogels allow recapitulation of both stiffness and dynamic stress-relaxing character of the native ECM. To date, dynamic hydrogels have been observed to influence cell fate when single cells were encapsulated, as observed with extended motor neurons axon bodies, 3D cell spreading and focal adhesion of hMSCs, and increased cartilage matrix formation by chondrocytes, but their application in aggregate or organoid culture is less studied.

Organoids derived from, for example, adult stem cells or induced pluripotent stem cells (iPSCs) mimic the organogenesis of the respective organ. In addition to their potential for studying development, disease modelling, and drug screening, organoids can be transplanted as a functional graft in patients. For example, kidney organoids may transplanted in patients with chronic kidney disease (CKD), which affects 11-13% of the population worldwide. Nevertheless, there are still many challenges to overcome before organoids are suitable for widespread clinical application. Many organoids resemble an immature developing organ at both the transcriptional and morphological levels, and prolonged culture does not improve their maturation. Moreover, for example in kidney organoids, morphological changes, an upregulation of off-target cell populations and aberrant ECM, containing increased types I and VI collagen and alpha smooth muscle actin (aSMA), are observed.

To further the field of organoid research and its potential application as transplants in patients, improved methods are needed that allow further or improved maturation of the organoid.

For example WO 2020/094776 A1, Hafeez et al. (Gels 2018, vol. 4, no. 4, page 85) and Sanchez-Moran et al. (Biomacromolecules 2019, vol. 20, no. 12, pages 4419-4429) broadly describe hydrogels, but none of these documents specify parameters defining properties of the hydrogel rendering it suitable for organoid culture.

These problems, among others, are overcome by the invention as described in the appended claims.

### SUMMARY OF THE INVENTION

In a first aspect, the invention relates to a method of generating a hydrogel for organoid culture, the method comprising:
providing a suspension of a polymer;
obtaining polymer with aldehyde groups; and
cross-linking the polymer with aldehyde groups with a cross-linking agent to obtain a cross-linked polymer; and
allowing the suspension comprising the cross-linked to form a hydrogel,
wherein the amount of oxidizing agent, the amount of cross-linking agent and amount of polymer are chosen such that the resulting hydrogel has a stiffness between 0.01 and 4 kPa as measured by shear moduli on a DHR2 rheometer from TA Instruments, and
   wherein the hydrogel has a stress relaxation time (t_{1/2}) of 10⁴ seconds or less as measured by relaxation modulus on a DHR2 rheometer from TA Instruments.

In a second aspect, the invention relates to a hydrogel obtained or obtainable by the method according to the first aspect of the invention.

In a third aspect, the invention relates to the hydrogel according to the second aspect of the invention comprising an organoid for use as a medicament.

In a fourth aspect, the invention relates to the hydrogel according to the second aspect of the invention comprising an organoid for use in a method of preventing, treating or ameliorating a disease, the use comprising transplanting the organoid in a subject in need thereof.

In a fifth aspect, the invention relates to the hydrogel according to the second aspect of the invention comprising a kidney organoid for use in a method of preventing, treating or ameliorating a kidney disease.

In a sixth aspect, the invention relates to the use of the hydrogel according to the second aspect of the invention in one or more of:
drug screening,
drug testing,
drug discovery,
drug development,
drug validation,
culturing cells, cell aggregates or organoids,
scale up for organoids,
disease modelling,
organoid implementation,
as a model organ.

In a seventh aspect the invention relates to a method for culturing an organoid, the method comprising the steps of:
culturing a cell, preferably a stem cell, induced pluripotent stem cell (iPSC), adult stem cell, embryonic stem cell, or progenitor cell, under conditions to allow an organoid to form,
embedding the organoid in or on the surface of a hydrogel obtained or obtainable by the methods according to claims 1 to 7; and
culturing the organoid in the hydrogel,
wherein the cross-linking step of the hydrogel is performed prior, during or after embedding the organoid in the hydrogel.

### BRIEF DESCRIPTION OF THE FIGURES

FIG. 1. Hydrogels of varying stiffness were designed. (a) Overview of iPSC differentiation and organoid generation. iPSCs were differentiated for 7 days, after which they were grown and matured as organoids for 14 days on the air-liquid interface (day 7+14). Organoids were then encapsulated in the different hydrogels (or left on the air-liquid interface as a control) and cultured for 4 subsequent days until day 7+18. (b) Schematic of the different hydrogel systems. All hydrogels were based on oxidised alginate cross-linked with oxime. Increasing the percentage (by weight) of oxidised alginate resulted in increased entanglement and binding sites, and therefore increased stiffness. Similarly, an increase in oxime increased the cross-linking density, and therefore increased the stiffness of the hydrogel. (c) The shear moduli (G′) of the three different hydrogel compositions (N=3, error bars representing standard deviation), of 20 kPa, 3 kPa and 0.1 kPa for the 4% alginate—20.2 μM oxime; 2% alginate—10.1 μM oxime, and 2% alginate—2.02 μM oxime hydrogels, respectively. The hydrogels had significantly different stiffnesses (one-way ANOVA, p<0.0001). (d) The stress relaxation of the different hydrogels compositions was similar (one-way ANOVA, p=0.32), with t_{1/2} values from 1.6-3.9×10⁴ s, despite their different stiffness.

FIG. 2. Kidney organoids formed in all dynamic hydrogels but renal cell types were absent in the 20 kPa hydrogel. Culture of the organoids for 4 days (from day 7+14 to 7+18) in hydrogels did not affect the presence of nephron segments in the 0.1 kPa (d) or 3 kPa (c) hydrogels when compared to organoids grown on an air-liquid interface until day 7+18 (a). Only the organoids encapsulated in the stiffest 20 kPa hydrogel (b) lacked loop of Henle segments (two left columns) and interstitial cells (two right columns). Staining for distal tubules (E-cadherin; ECAD); the loop of Henle (NKCC2; SLC12A1); proximal tubules (lotus tetragonolobus lectin; LTL); glomeruli (nephrin; NPHS1); and interstitial cells (homeobox protein Meis 1/2/3; MEIS1/2/3) as indicated. DAPI staining (blue) for nuclei. The white box denotes the area of interest enlarged in the respective right panel. Scale bars: 50 μm. Representative images of N=3 organoid batches with n=3 organoids per batch.

FIG. 3. Evidence of epithelial-mesenchymal transition (EMT) observed in organoids encapsulated in the 3 and 20 kPa hydrogels. (a) Analysing single-cell RNA sequencing datasets from literature showed an increase of number of cells that expressed the EMT-related marker (TWIST1) and mesenchymal markers (VIM, ACTA2, SNAI1, and CDH2) and a reduced number of cells with the epithelial marker (CDH1). (b-i) Encapsulated organoids until day 7+18 showed an expression of the EMT transition marker SNAIL (green, two left columns) in the 20 kPa (c) and 3 kPa (d) hydrogel, whereas no expression was detected in the 0.1 kPa hydrogel (e) or the air-liquid interface control (b). Moreover, higher expression of the mesenchymal marker vimentin (red, two right columns) was observed in the 20 kPa (g) and 3 kPa (h) hydrogels compared to the air-liquid interface (f). Staining for distal tubules (E-cadherin; ECAD); proximal tubules (lotus tetragonolobus lectin; LTL); and fibroblasts (alpha smooth muscle actin; αSMA) as indicated. DAPI staining (blue) for nuclei. The white box denotes the area of interest enlarged in the respective right panel. Scale bars: 50 μm. Representative images of N=3 organoid batches with n=3 organoids per batch.

FIG. 4. Fast-relaxing dynamic hydrogel further reduced the undesired EMT-related phenotype (a) Schematic of two alginate hydrogels with different cross-linkers—hydrazone or oxime—to influence stress relaxation times. (b) The stiffness of both hydrogels is approximately 0.1 kPa (0.08±0.05 and 0.07±0.05 kPa for 2.02 μM oxime and 10.1 μM hydrazone, respectively; t-test, p<0.0001). (c) The stress relaxation of the fast-relaxing hydrazone hydrogel is an order of magnitude faster, with a value of 4.1×10³ s, compared to the 3.9×10⁴ s for the soft 0.1 kPa oxime hydrogel (d-i) Encapsulated organoids until day 7+18 in the fast stress-relaxing hydrogel (f, i) showed less staining for aSMA (red, two left columns) and vimentin (red, two right columns) compared to those grown in the slow-relaxing hydrogel (e, h) and on the air-liquid interface (d, g). Staining for distal tubules (E-cadherin; ECAD); proximal tubules (lotus tetragonolobus lectin; LTL); and the mesenchymal markers (Zinc finger protein SNAI1; SNAIL and Vimentin), as indicated. DAPI staining (blue) for nuclei. The white box denotes the area of interest enlarged in the respective right panel. Scale bars: 50 μm. Representative images of N=3 organoid batches with n=3 organoids per batch.

FIG. 5. Stiffness and stress relaxation affect the polarity of the tubule lumen structures, ciliary length and frequency. Encapsulated organoids until 7+18 d in 0.1 kPa hydrogels (d,i,n,s and e,j,o,t) showed apical enrichment of LTL⁺ tubules compared to organoids grown in the other hydrogels. The air-liquid interface grown organoid show a clear apical and basal orientated LTL⁺ staining (a.f,k,p). When comparing LTL intensity apical vs basal a significant increase expression (p=0.0002 and p<0.0001 for slow and fast-relaxing hydrogel respectively) was observed in the soft hydrogels. Intensity plots (k-o) show the LTL intensity distribution over the dotted lines. The heat maps show overall LTL staining distribution over complete tubule structure. DAPI staining (blue) for nuclei. The white box denotes the area of interest enlarged in the respective right panel. Scale bars: 50 μm. Representative images of N=3 organoid batches with n=3 organoids per batch. v) Primary cilia length were observed to vary in length in the different hydrogels compared to the air-liquid interface. Representative images of N=3 organoid batches with n=3 organoids per batch. w) When measuring cilia length for cells with positive LTL staining, we observed significantly longer primary cilia for the 0.1 kPa fast hydrogel (***<0.0005; one-way ANOVA), compared to the air-liquid interface. N=3 organoid batches per condition, 5 images per batch, 40 cilia of LTL⁺ cells were measured per condition. x) Cilia frequency was altered in the different hydrogels compared to the air-liquid interface. N=3 organoid batches per condition, 3 images per condition (ns=non-significant, *<0.05, ***<0.001, one-way ANOVA).

FIG. 6. All renal cell types and reduced collagen type 1a1 were observed in the kidney organoids encapsulated in the 0.1 kPa, fast-relaxing hydrogel. Immunohistochemistry of kidney organoids encapsulated in the fast-relaxing hydrogel after 7+18 d, staining for the proximal tubules (LTL, lotus tetragonolobus lectin, in yellow), distal tubules (E-cadherin; ECAD, in green in a), loop of Henle (NKCC2; SLC12A1, in red in b), interstitial cells (homeobox protein Meis 1/2/3; MEIS1/2/3, in red in c), glomeruli (nephrin; NPHS1, in green in d), type 1a1 collagen (green in e) and type 6a1 collagen (red in f). DAPI staining (blue) for nuclei. Single channels are shown in the two left columns; merged images in the two right columns. The white box denotes the area of interest enlarged in the respective panel to the right. Scale bars: 50 μm. Representative images of N=3 organoid batches with n=3 organoids per batch.

### DETAILED DESCRIPTION

Pluripotent stem cell-derived organoids offer a promising solution for implantation in patients, yet current organoid protocols often lead to off-target cells and phenotypic alterations, preventing advancement. For example, kidney organoids can be used in patients suffering from renal failure. Here, we created various dynamic hydrogel architectures, conferring a controlled and biomimetic environment for organoid encapsulation. We investigated how hydrogel stiffness and stress relaxation affect renal phenotype and undesired fibrotic markers. We observed stiff hydrogel encapsulation led to an absence of certain renal cell types and signs of an epithelial-mesenchymal transition (EMT), whereas encapsulation in soft-stress-relaxing hydrogels led to all major renal segments, fewer fibrosis/EMT associated proteins, apical proximal tubule enrichment, and primary cilia formation, representing a significant improvement over current approaches to culture organoids such as for example kidney organoids. Our findings show that engineering hydrogel mechanics and dynamics has a decided benefit for organoid culture. These structure-property-function relationships enable rational design of materials, allowing functional engraftments and disease-modelling applications.

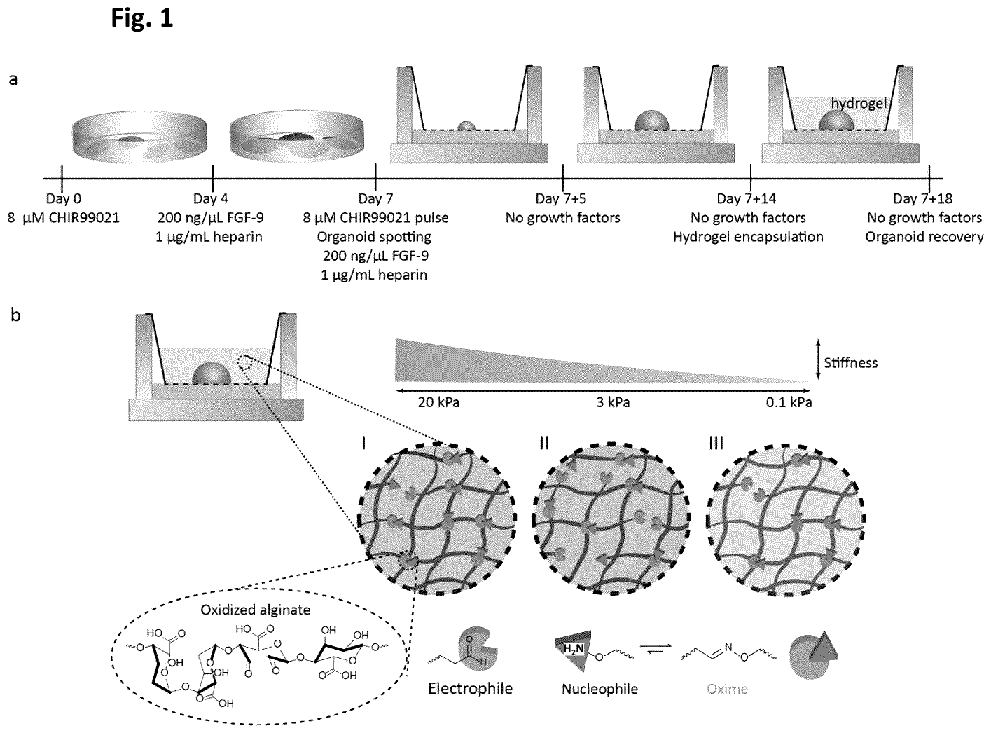

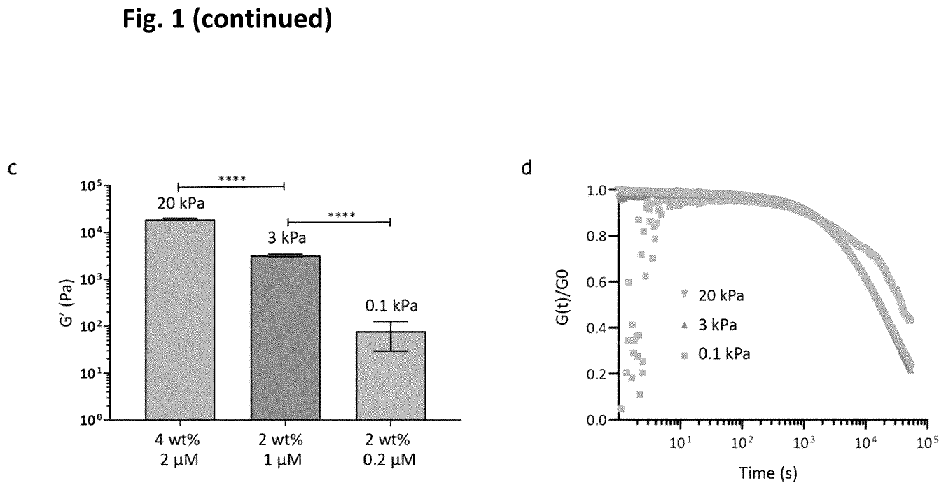

Here, the inventors used kidney organoids as a representative model and tested the influence of dynamic hydrogels on kidney organoids by designing a small hydrogel library based on oxidised alginate: three hydrogels of tuneable stiffness (ranging from 0.1 to 20 kPa), and two soft hydrogels (0.1 kPa) with different stress relaxation (slow and fast). They used an imine-type dynamic covalent cross-linking possessing a range of equilibrium constants (K_{eq}) that affect the hydrogel stiffness and tuneable hydrolysis rates (k⁻¹) that change the rate of cross-link rearrangement and stress relaxation. Kidney organoids cultured until day 7+14 (7 days of iPSC differentiation and 14 days of organoid culture on an air-liquid interface) were encapsulated in these hydrogels and cultured for 4 subsequent days (FIG. 1a). They confirmed the formation of renal structures by immunohistochemistry. They investigated the expression of off-target ECM and EMT-related markers, as well as variations in the formation of lumen and cilia structures in renal organoids cultured in hydrogels of different stiffness and stress relaxation. Soft hydrogels with fast relaxation properties resulted in more mature kidney organoids, determined by the above parameters, and compared to the stiffer hydrogels or slow-relaxing hydrogels. Their findings reinforce the concept of carefully selecting not only the stiffness, but also stress-relaxation properties of encapsulating matrices and highlight the potential of tuning hydrogel properties to influence organoid cultures.

Therefore, in a first aspect the invention relates to a method of generating a hydrogel for organoid culture, the method comprising:
providing a suspension of a polymer;
obtaining polymer with aldehyde groups; and
cross-linking the polymer with aldehyde groups with a cross-linking agent to obtain a cross-linked polymer; and
allowing the suspension comprising the cross-linked to form a hydrogel,
wherein the amount of oxidizing agent, the amount of cross-linking agent and amount of polymer are chosen such that the resulting hydrogel has a stiffness between 0.01 and 4 kPa as measured by shear moduli on a DHR2 rheometer from TA

Instruments, and wherein the hydrogel has a stress relaxation time (t_{1/2}) of 10⁴ seconds or less as measured by relaxation modulus on a DHR2 rheometer from TA Instruments. When used herein, the term hydrogel refers to a three-dimensional (3D) network of hydrophilic polymers that can swell in water and hold a large amount of water while maintaining the structure due to chemical or physical cross-linking of individual polymer chains.

When used herein the term organoid refers to a miniaturized and simplified version of an organ produced in vitro in three dimensions that shows microanatomy with some resemblance to the native tissue. Generally derived from one or a few cells from a tissue, embryonic stem cells or induced pluripotent stem cells, which can self-organize in three-dimensional culture owing to their self-renewal and differentiation capacities. The organoid may be derived from human cells or non-human animal cells.

When used herein the term to culture when referring to organoids is intended to mean to keep an organoid in culture in vitro, preferably further growing or expanding the organoid and/or maturing the organoid to a more mature organ-like state.

The present invention is based on the insight that the stiffness of the hydrogel encapsulating the organoid has a profound effect on the development and growth of the organoid. The inventors found that a too stiff hydrogel e.g. 20 kPa prevents some cell types from forming compared with less stiff hydrogels or organoids grown on liquid air-interface. Therefore, the stiffness is preferably 10 kPa or less, more preferably 4 kPa or less, even more preferably 1 kPa or less.

The inventors further found that by further reducing the stiffness of the hydrogel, epithelial to mesenchymal transition (EMT) may be avoided or reduced when compared to more stiff hydrogels or when compared to organoids grown on liquid air-interface. Therefore the stiffness of the hydrogel is preferably less than 5 kPa, such as less than 4, 3, 2, 1, 0.9, 0.8, 0.7, 0.6, 0.5, 0.4, 0.3 or less than 0.2 kPa. Therefore, in an embodiment the stiffness is between 0.01 and 1 kPa, more preferably between 0.01 and 0.5 kPa, as measured by shear moduli on a DHR2 rheometer from TA Instruments.

Stiffness and relaxation times of a hydrogel can be determined using rheology according to protocols known to the skilled person. For example, rheological characterisation on the hydrogels can be performed on a DHR2 rheometer from TA Instruments. According to an exemplary protocol: Time sweeps of preformed hydrogels are taken over 360 s with an 8 mm parallel plate geometry at 20° C. with an applied strain of 1% at 3.14 rad/s. During loading, the gap size is adjusted to achieve 0.1 N of normal force. The shear modulus (G′) for a given sample is taken to be the mean recorded value with a minimum of three sample replicates per formulation. Stress relaxation measurements are performed using a 20 mm cone-plate geometry equipped with solvent trap. Time sweeps are measured over 3.5-9 h (maintained at 20° C.) to monitor crosslinking progress with an applied strain of 1% at 10 rad/s. Once a plateau is reached, a frequency sweep is performed from 100-0.1 rad/s with an applied strain of 1% and 10 pts/dec. Finally, to measure the stress relaxation behaviour, the relaxation modulus is monitored over 15.5 h with an initial applied strain of 20% maintained over the course of the measurement.

The inventors used sodium alginate as a proof of principle, it is however envisioned that any biocompatible polymer from which properties such as stiffness can be tuned is suitable for use in the invention. Therefore in an embodiment the polymer is sodium alginate, hyaluronic acid, PEG, PEG derivatives, gelatin, GelMA, dextran, PVA, PAA, PAcm, collagen, or peptide modified polymers thereof or mixtures thereof.

The inventors further show that EMT in organoids, as measured by marker proteins, can be further reduced by decreasing the stress relaxation time of the hydrogel. Therefore in an embodiment, the hydrogel is a fast relaxing hydrogel, preferably wherein the hydrogel has a stress relaxation time of 10⁴ seconds or less, for example 0.9×10⁴, 0.8×10⁴, 0.7×10⁴, 0.6×10⁴, or 0.5×10⁴ or less, as measured by relaxation modulus on a DHR2 rheometer from TA Instruments with 20% strain.

The relaxation time can for example be determined as described above.

It is understood that aldehyde groups in the polymer may be obtained for example by reacting the polymer with oxygen. Alternatively protected aldehyde groups can be used and the protecting group can be cleaved off, or aldehyde groups can be added to the polymer. Alternatively an oxidizer may be used to oxidise the hydrogel (e.g. the alginate) to obtain aldehyde groups for imine-type cross-linking, which allows for dynamic reshuffling of the cross-links in cell culture conditions. Therefore increasing the amount of oxidiser increases the amount of crosslinking in the final product, resulting in higher stiffness of the crosslinked hydrogel. Vice versa reducing the amount of oxidiser reduces the amount of crosslinking and subsequently the stiffness of the crosslinked hydrogel. For example, the amount of crosslinker may be chosen based on the theoretical degree of oxidation of the hydrogel, for example between 0.1 and 20% theoretical degree of oxidation, more preferably between 1 and 10% theoretical degree of oxidation. It is understood that any oxidising agent may be used that is able to form an aldehyde group on the hydrogel biomolecules. Thus, in an embodiment the invention relates to a method of generating a hydrogel for organoid culture, the method comprising: providing a suspension of a polymer; oxidizing the polymer with an oxidizing agent to obtain polymer with aldehyde groups; and cross-linking the polymer with aldehyde groups with a cross-linking agent to obtain a cross-linked polymer; and allowing the suspension comprising the cross-linked to form a hydrogel, wherein the amount of oxidizing agent, the amount of cross-linking agent and amount of polymer are chosen such that the resulting hydrogel has a stiffness between 0.01 and 4 kPa as measured by shear moduli on a DHR2 rheometer from TA Instruments and wherein the hydrogel has a stress relaxation time (t_{1/2}) of 10⁴ seconds or less as measured by relaxation modulus on a DHR2 rheometer from TA Instruments. In an embodiment, the oxidizing agent is an oxygen-atom transfer (OAT) agent, preferably wherein the oxygen-atom transfer (OAT) agent selected from a periodate salt such as NaIO₄, a permanganate salt such as KMnO₄, H₂O₂, a chromate salt, OsO₄, a perchlorate salt or combinations thereof.

Similarly, it is understood that the crosslinker reacts with the aldehyde to form an imine. Therefore the crosslinking agent preferably is a compound with at least two amine groups available to react with an aldehyde (or ketone) so that the cross-linking agent can connect (crosslink) two different molecules (or from an intramolecular crosslink). Therefore, in an embodiment the cross-linking agent is a compound having at least two amine groups, preferably wherein the cross-linking agent is selected from the group consisting of
an alkoxy compound having formula (I):

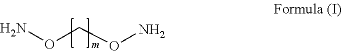

wherein m is 2 to 12,
a semicarbazide compound having formula (II):

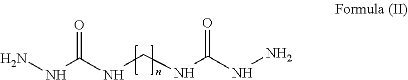

wherein n is 2 to 12, and
a hydrazide compound having formula (III):

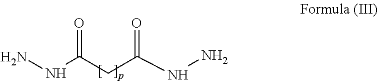

wherein p is 2 to 12
wherein

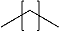

represents an alkylene group having m, n or p carbon atoms, wherein 1 or more carbon atoms can be replaced by a heteroatom selected from O, S and N;
a compound having formula (IV):

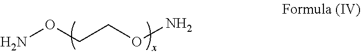

wherein x is 1 to 250;
a compound having formula (V):

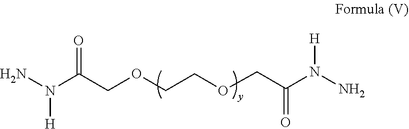

wherein y is 1 to 250;
a compound having formula (VI):

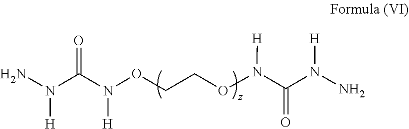

wherein z is 1 to 250;
a compound having formula (VII)

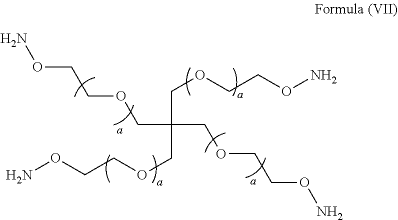

wherein each a is individually 1 to 250;
a compound having formula (VIII)

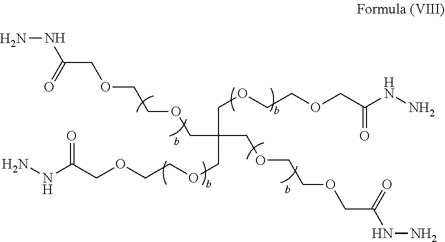

wherein each b is individually 1 to 250;
a compound having formula (IX):

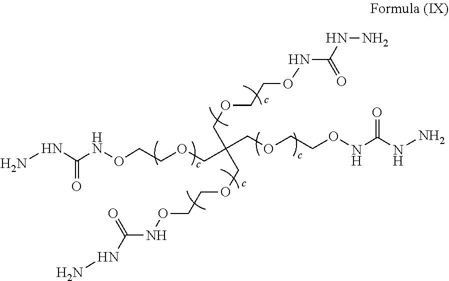

wherein each c is individually 1 to 250;
a compound having formula (X):

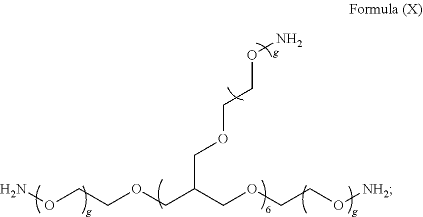

wherein each g is individually 1 to 250
a compound having formula (XI)

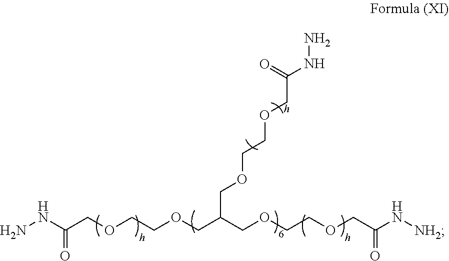

wherein each h is individually 1 to 250
a compound having formula (XII):

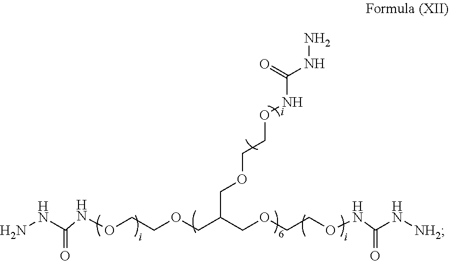

wherein each i is individually 1 to 250
a compound having formula (XIII):

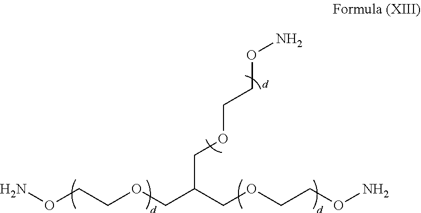

wherein each d is individually 1 to 250;
a compound having formula (XIV)

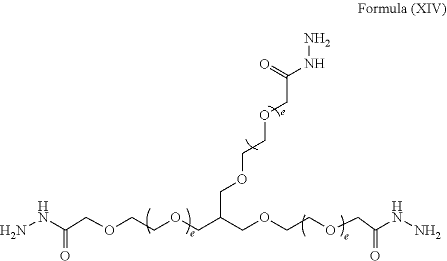

wherein each e is individually 1 to 250;
a compound having formula (XV):

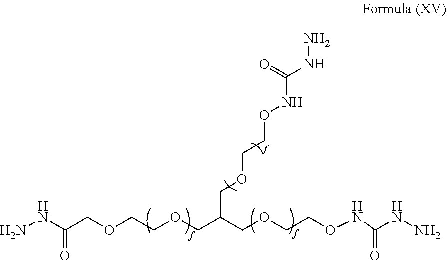

wherein each f is individually 1 to 250;
a compound having formula (XVI):

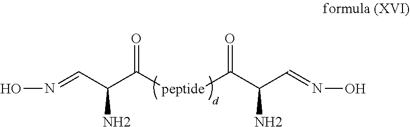

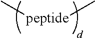

wherein d represents a peptide of 2 to 45 amino acids, preferably an enzymatically cleavable peptide; and
a compound having formula (XVII):

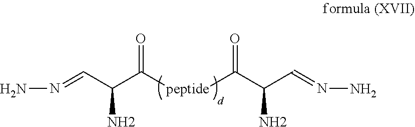

wherein

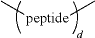

represents a peptide of 2 to 45 amino acids, preferably an enzymatically cleavable peptide;
preferably wherein the crosslinker is O,O′-1,3-propanediylbishydroxylamine or adipic dihydrazide.

The amount of crosslinking agent added can for example be between 0.2 and 200 μM, preferably between 0.5 and 100 μM more preferably between 1 and 80 μM.

It is further understood that the stiffness of the crosslinked hydrogel is further influenced by the concentration of the hydrogel. Therefore, the amount of hydrogel may for example be chosen to be between 0.2 and 10 w/v %, preferably between 0.5 and 5 w/v % more preferably between 1 and 5 w/v %, however it is understood that the percentage depends on the chosen polymer. For example when using PEG based polymers over 10 w/v % may be needed, or for alginate it depends whether low or high molecular weight alginate is used. The skilled person is able to adapt the weight percentage based on the chosen polymer to obtain the desired stiffness.

It is further envisioned that the method according to the first aspect of the invention is used in culturing an organoid. Therefore in an embodiment, the method is further used to culture an organoid or cell aggregate, the method further comprising the steps of:
culturing a cell, preferably a stem cell, induced pluripotent stem cell (iPSC), adult stem cell, embryonic stem cell, or progenitor cell, under conditions to allow an organoid or cell aggregate to form,
embedding the organoid or cell aggregate in or on the surface of the polymer suspension; and
culturing the organoid or cell aggregate in the hydrogel,
wherein the cross-linking step is performed on the oxidized polymer prior, during or after embedding the organoid or cell aggregate.

Methods for culturing stem cells, iPSC or progenitor cells to form organoids are known to the person skilled in the field. For example in kidney organoids, the cells are grown under differentiating conditions by adding organ type dependent factors to the culture medium. After some time the cells are placed on air-liquid interface for maturation. In most protocols, the organoids will remain in air-liquid interface. In the methods disclosed herein, the organoid is kept on air-liquid interface for only a limited time, e.g. 4, 5, 6, 7, 8, 9, 10, 11, 12, 13 or 14 days, or more, and then encapsulated in or put on the surface of the hydrogel as described herein.

The method describes that the crosslinking step takes place prior to, during or after embedding the organoids in the hydrogel or placing the organoids on the surface of the hydrogel. Crosslinking prior to embedding may be achieved for example by mixing oxidized hydrogel stock solution with stem cell differentiation medium comprising the crosslinking agent to the desired concentration—allowing crosslinked hydrogel to form—and covering the organoids with the crosslinked hydrogel to embed them. Once embedded in the hydrogel the organoids can be cultured using normal protocols.

In an embodiment the organoid or cell aggregate is a kidney organoid, an intestinal organoid, a pancreatic organoid, a neural organoid, a hepatic organoid, a thyroid organoid, a stomach organoid, an ovarian organoid, a prostate organoid, a splenic organoid, oesophageal organoid, a breast organoid, a bladder organoid, a lung organoid, an optic organoid, an inner ear organoid, a cardiac organoid, a biliary organoid, a salivary gland organoid, a pituitary gland organoid, a lymphoid organoid, a bladder organoid, a tongue organoid, a cerebral/brain organoid, a spinal cord organoid, a fallopian tube organoid, a lacrimal gland organoid, a skin organoid, and a hippocampal organoid, or wherein the cell aggregate are mesenchymal stem cells or cells or progenitor derived thereof such a osteoblasts, osteoclasts, chondrocytes or adipocytes.

When used herein the term organoid refers to a cell aggregate having at least two distinct, preferably more, specialized cell types generally present on an organ or tissue, which is grown in culture from one or more stem cells, adult stem cells, induced pluripotent stem cells or progenitor cells. The specialized cell types may be partially or fully differentiated stem cells or progenitor cells, thus the organoid does not necessarily represent a mature model of the organ or tissue but may also be representative of the organ or tissue in a state of development.

When used herein, the term cell aggregate refers to a plurality of cells in close association, wherein the cells may be phenotypically the same or different, and wherein if the cell types are phenotypically different these phenotypes do not necessarily represent the same organ or tissue type.

In a second aspect, the invention relates to a hydrogel obtained or obtainable by the method according to the first aspect of the invention. It is further envisioned that the invention relates to a kit of parts comprising a stock solution of oxidized hydrogel, for example oxidized sodium alginate and a stock solution of crosslinking agent, wherein the stock solutions are such that the concentration is as intended after mixing. For example the stock solutions may be 2× and mixed in 1:1 ratio to obtain a 1× final solution of components (the oxidized hydrogel and crosslinking agent).

The thus obtained hydrogel may have one or more organoids embedded. Therefore, in an embodiment the invention relates to hydrogel obtained or obtainable according to the method of the first aspect of the invention further comprising an organoid or cell aggregate, wherein the organoid or cell aggregate is a kidney organoid, an intestinal organoid, a pancreatic organoid, a neural organoid, a hepatic organoid, a thyroid organoid, a stomach organoid, an ovarian organoid, a prostate organoid, a splenic organoid, oesophageal organoid, a breast organoid, a bladder organoid, a lung organoid, an optic organoid, an inner ear organoid, a cardiac organoid, a biliary organoid, a salivary gland organoid, a pituitary gland organoid, a lymphoid organoid, a bladder organoid, a tongue organoid, a cerebral/brain organoid, a spinal cord organoid, a fallopian tube organoid, a lacrimal gland organoid, a skin organoid, and a hippocampal organoid, or wherein the cell aggregate are mesenchymal stem cells or cells or progenitor derived thereof such a osteoblasts, osteoclasts, chondrocytes or adipocytes.

In a particularly preferred embodiment the organoid is a kidney organoid.

The hydrogels comprising organoids as described herein find use for example for research purposes or drug screening, as the organoids resemble a more mature organ compared to traditionally prepared organoids. Alternatively the in hydrogel embedded organoids as described herein may be transplanted directly in a patients as functional grafts. Therefore, in a third aspect the invention relates to the hydrogel comprising an organoid according to the second aspect of the invention for use as a medicament.

Particularly the invention relates to the hydrogel comprising an organoid according to the second aspect of the invention for use in a method of preventing, treating or ameliorating a disease, the use comprising transplanting the organoid in a subject in need thereof.

Non-limiting examples of a disease are kidney disease such as chronic kidney disease (kidney organoid), short bowel syndrome (intestinal organoid), cystic fibrosis (in multiple organs), cancer (in multiple organs) or diabetes (pancreatic organoids or beta cell aggregates).

In a preferred embodiment the invention relates to the hydrogel comprising a kidney organoid according to the second aspect of the invention for use in a method of preventing, treating or ameliorating a kidney disease. Preferably, the kidney disease is chronic kidney disease.

It is further considered that the hydrogels described herein comprising organoids or cell aggregates may be particularly useful in the screening, testing and validation of drugs, or can serve as improved model systems for organs or tissues for research and development applications. Therefore, in a further aspect the invention relates to the use of the hydrogel according to the second aspect of the invention in one or more of:
drug screening,
drug testing,
drug discovery,
drug development,
drug validation,
culturing cells, cell aggregates or organoids,
scale up for organoids,
disease modelling,
organoid implementation,
as a model organ. In a particularly preferred embodiment the hydrogel is used to culture organoids.

It was found that the presently described hydrogels are particularly useful for culturing organoids. Therefore in a further aspect the invention relates to a method for culturing an organoid, the method comprising the steps of:
culturing a cell, preferably a stem cell, induced pluripotent stem cell (iPSC), adult stem cell, embryonic stem cell, or progenitor cell, under conditions to allow an organoid to form,
embedding the organoid in or on the surface of a hydrogel obtained or obtainable by the methods according to the first aspect of the invention; and
culturing the organoid in the hydrogel,
wherein the cross-linking step of the hydrogel is performed prior, during or after embedding the organoid in the hydrogel.

The skilled person will appreciate that the timing for encapsulating the organoid in the hydrogel depends among others on the protocol used. For example, the inventors use a protocol where iPSCs are differentiated in culture for 7 days, after which the cells are grown and matured as organoids for 14 days (herein indicated as 7+14 days) on an air liquid interface prior to encapsulating the organoids in the hydrogel. It is however understood that other protocols may be used, for an overview see for example Koning 2020 (Cellular and Molecular Life Sciences 77 (9731)). Therefore the organoid may for example be encapsulated 7, 8, 9, 10, 11, 12, 13, 14, 15, 16, 17, 18, 19, 20, 21, 22, 23, 24, 25, 26, 27, 28, 29 or 30 days after onset of the differentiation of the stem cell, induced pluripotent stem cell (iPSC) or progenitor cell. Protocols for differentiating stem cells, induced pluripotent stem cells (iPSCs) or progenitor cells are known to the skilled person, see e.g. Koning 2020 and references therein.

As described herein in the below examples, the hydrogels described herein having a a stiffness between 0.01 and 3 kPa and a stress relaxation time (t_{1/2}) of 10⁴ seconds or less allow for the culture of organoids which present more cell types, display less undesirable EMT phenotypes, and can be kept in culture longer compared to using known methods.

### EXAMPLES

### Hydrogels were Designed with Varying Stiffness

To determine the role of hydrogel stiffness on renal organoid phenotype and ECM deposition, we designed three alginate hydrogels with varying stiffness (FIG. 1b). We selected sodium alginate, a naturally-derived, biocompatible, and non-adhesive biomaterial, which is biodegradable when oxidised and has been used in FDA-approved applications. Sodium alginate can form an ECM-like hydrogel and has previously been shown to support culture of kidney organoids. We oxidised the alginate to obtain aldehyde groups for imine-type cross-linking, which allows for dynamic reshuffling of the cross-links in cell culture conditions.

Sodium alginate was oxidised using sodium periodate (NaIO₄) at a 10% theoretical degree of oxidation, then characterised by ¹H-NMR and gel permeation chromatography (GPC; Table 2). As a cross-linker, we used a small bifunctional oxime (O,O′-1,3-propanediylbishydroxylamine). Starting at 2 wt % oxidised alginate, increasing the concentration of bifunctional oxime increased the cross-linking density, which subsequently resulted in an increased stiffness, ranging from 0.1 to 3 kPa for 2.02 to 10.1 μM of oxime cross-linker, respectively (FIG. 1b-c and S3a-c). To obtain a higher stiffness, we increased the weight percentage of the oxidised alginate (from 2 to 4 wt %) while keeping the 1:1 oxime to aldehyde ratio (20.2 μM oxime cross-linker), which resulted in a hydrogel with a stiffness of 20 kPa (FIGS. 1c and S3a-c). While the stiffness of the hydrogels was significantly different in each composition (p<0.0001), the characteristic stress relaxation times (t_{1/2}, defined as the time taken for the relaxation modulus to reach half of its initial value) were similarly long, with values from 1.6-3.9·10⁴ s (p=0.32, FIG. 1d)

### Kidney Organoids Formed in All Hydrogels but Renal Cell Types were Absent in the 20 kPa Hydrogel

Kidney organoids were cultured until day 7+14, at which point they were encapsulated in the different hydrogels for 4 additional days of culture (FIG. 1a). The starting point of day 7+14 was selected because our previous work demonstrated that an overexpression of type 1a1 collagen began at day 7+14 of organoid culture and could be significantly reduced by encapsulating the organoids in a hydrogels for 4 additional days¹¹. The hydrogel solutions were added on top of the organoid on the air-liquid interface, and hydrogel cross-linking was observed after approximately 1 h incubation at 37° C. After 4 d (day 7+18), the hydrogels were removed, and organoids were stained with calcein AM and EthD-1 to assess live/dead cells. We detected no difference in the morphology of the organoids (bright-field microscopy) or live/dead cells compared to organoids cultured on the air-liquid interface at day 7+18.

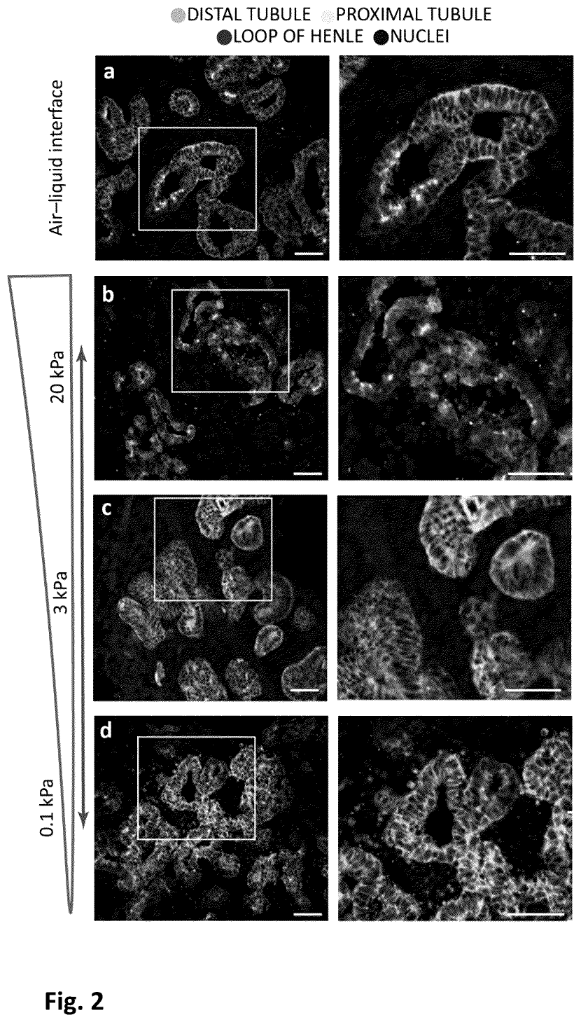

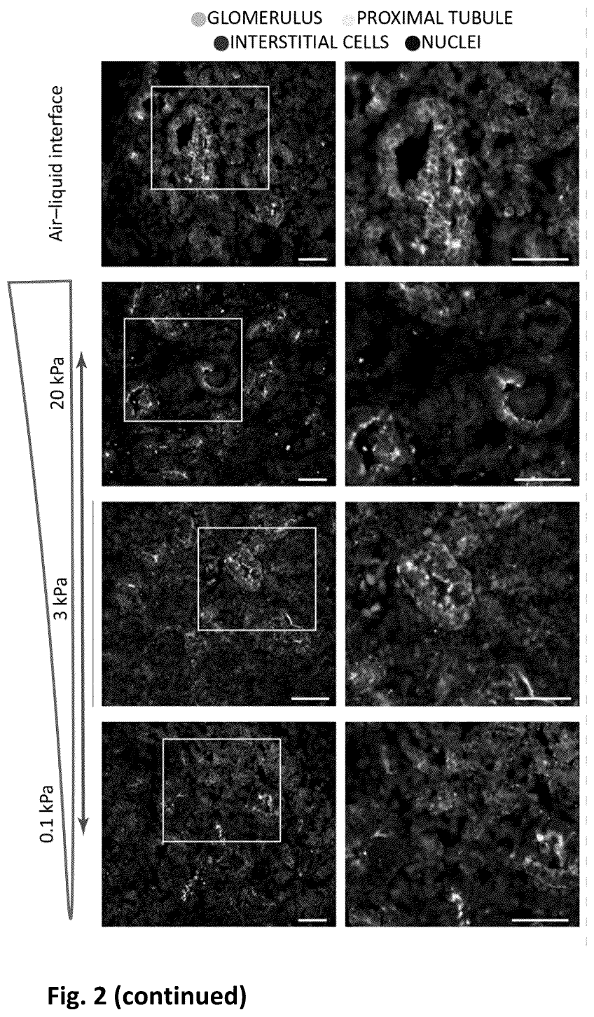

To determine the effect of the hydrogel stiffness on the presence of different renal segments, the organoids were stained with relevant markers for distal tubules (E-cadherin; ECAD), glomeruli (nephrin; NPHS1), interstitial cells (homeobox protein Meis 1/2/3; MEIS1/2/3), loop of Henle (NKCC2; SLC12A1), and proximal tubules (LTL). The organoids recovered from the 0.1 and 3 kPa hydrogel possessed all expected segments (FIG. 2c-d) and had no distinguishable differences from organoids cultured on the air-liquid interface (FIG. 2a). However, organoids encapsulated in the stiffer 20 kPa hydrogel lacked interstitial and loop of Henle cells, and showed diminished lumen structures (LTL) and distal tubules (ECAD) (FIGS. 2b and S5b) compared to organoids cultured on both the air-liquid interface and other hydrogels (0.1 and 3 kPa).

### No Epithelial-Mesenchymal Transition (EMT) Observed in Organoids Encapsulated in the Soft 0.1 kPa Hydrogels

Because previous work demonstrated that prolonged (>7+14 d) organoid culture led to protein expression indicating fibrosis, namely collagens 1a1 and 6a1 (Geuens T, et al. Biomaterials 2021, 275, 120976. https://doi.org/10.1016/j.biomaterials.2021.120976), we wished to determine whether the kidney organoids encapsulated in the hydrogels showed signs of fibrosis.

Immunohistochemistry data showed a reduction of type 1a1 collagen in organoids encapsulated in all hydrogels compared to the air-liquid interface control. In contrast, the expression of type 6a1 collagen was unchanged, indicating that the hydrogel stiffness had a selective modifying effect on the type 1a1 collagen.

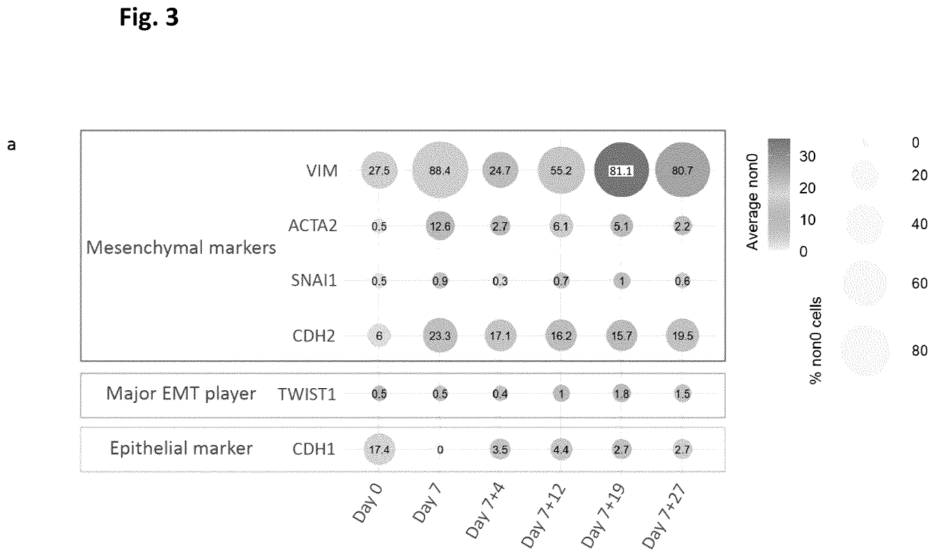

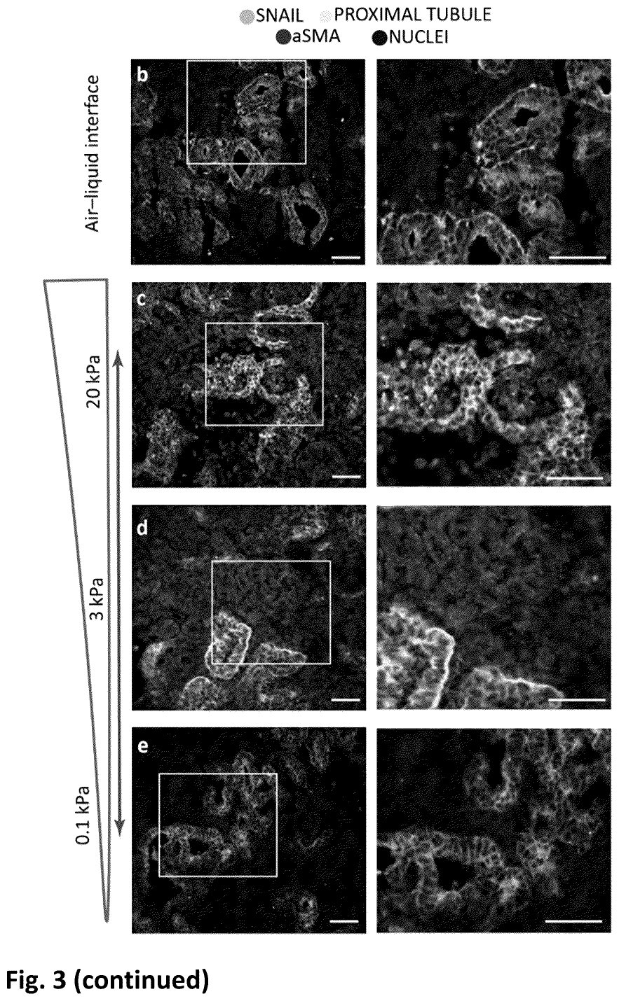

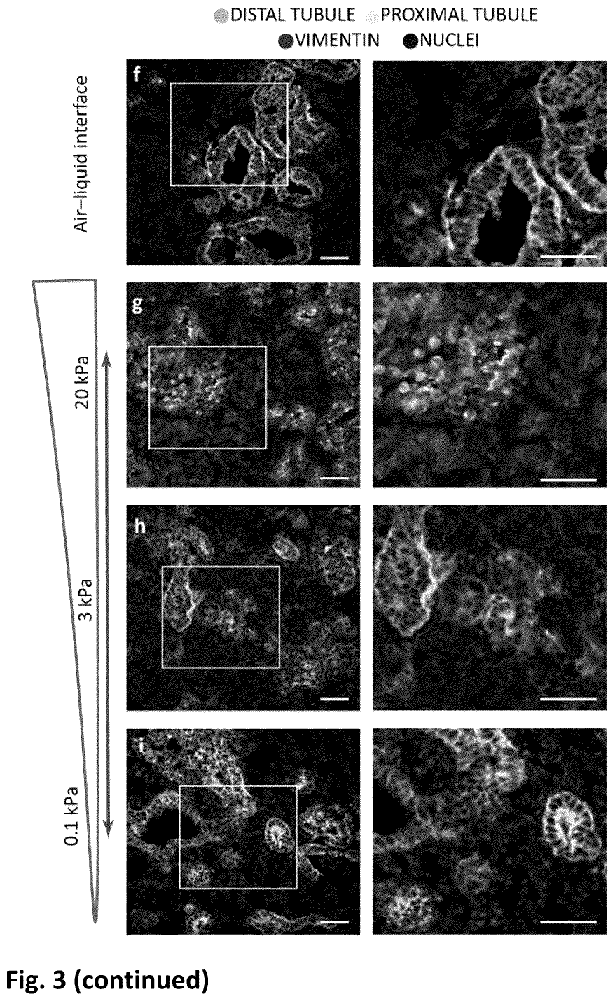

Since EMT is an early marker of renal fibrosis, we also analysed the expression of EMT markers to determine the influence of hydrogel stiffness. We began by analysing single-cell RNA sequencing datasets from literature of kidney organoids culture up to day 7+27 (Wu H, et al. Cell Stem Cell 2018, 23(6): 869-881.) for EMT-related markers. We found the following indicators of an EMT: upregulation of twist family bHLH transcription factor 1 (TWIST1), snail zinc-finger transcriptional factor 1 (SNAI1encoding SNAIL), N-cadherin (CDH2), aSMA (ACTA2), and vimentin (VIM), and the downregulation of E-cadherin (CDH1) (FIG. 3a). These EMT markers were upregulated from day 7+12 and found in all cells cultured at day 7+27 on the air-liquid interface, especially the mesenchymal marker vimentin; while the epithelial marker E-cadherin was downregulated.

For organoids cultured in the hydrogels up to 7+18 days, immunohistochemistry showed the classic EMT markers in the 3 and 20 kPa hydrogels. SNAIL-positive cells were found in organoids cultured in the 3 kPa (FIGS. 3d) and 20 kPa (FIG. 3c) hydrogels, but not in the 0.1 kPa hydrogel (FIG. 3e) or air-liquid interface (FIG. 3b). Organoids cultured in the two stiffer hydrogels (3 and 20 kPa; FIG. 3d and c) expressed more aSMA than organoids cultured in the 0.1 kPa hydrogel (FIG. 3e) and the air-liquid interface (FIG. 3b). Expression of the mesenchymal marker vimentin was observed in all organoids (FIG. 3f-i), with higher expression in the organoids in the 3 and 20 kPa hydrogels (FIG. 3h and g). Vimentin expression establishes the presence of mesenchymal cells and is required for EMT-related renal fibrosis, where it is upregulated in proximal tubular cells when repair is initiated after epithelial injury. In all organoids, vimentin did not co-stain with the proximal tubule and distal tubule cells, but was located between these segments (FIG. 3f-i and S8f-i). These findings, together with the absence of interstitial (MEIS1/2/3⁺) cells, (FIG. 2b), increased aSMA-positive cells (FIG. 3c), reduced lumen structures, and diminished distal tubules (FIG. 2b) in the stiffest 20 kPa hydrogel, suggest some epithelial cells have turned to mesenchymal cells and are contributing to the organoid stroma.

Our observations of fibrogenesis in organoids cultured on stiffer hydrogels is consistent with the literature reporting a link between fibrosis and tissue stiffness. For example, the kidney becomes stiffer (>15 kPa) with increasing fibrosis. The stiff hydrogel of >20 kPa in this study may activate the EMT pathways leading to fibrosis. Ondeck et al. (Proceedings of the National Academy of Sciences of the United States of America 2019, 116(9): 3502-3507.) saw a similar trend of increased EMT markers TWIST and SMAD2/3 in mammalian epithelial cells when dynamically increasing the stiffness of a methacrylate glycosaminoglycan hyaluronic acid hydrogel from 0.1 kPa to 3000 kPa. Others have reported that a softer environment can accelerate the differentiation of iPSC derived kidney organoids (Garreta E, et al. Nat Mater 2019, 18(4): 397-405) and can prime undifferentiated cells to lineage commitment (Ahmed K, et al., PLOS ONE 2010, 5(5): e10531). Moreover, Chen et al. (American Journal of Physiology-Renal Physiology 2014, 307(6): F695-F707) observed the prevention of TGF-β1-induced EMT when porcine kidney proximal tubule cells were cultured on collagen type 1-coated polyacrylamide gels of ˜0.2 kPa stiffness, while cells on a stiffer matrix (>0.7 kPa) highly expressed mesenchymal markers. Indeed, we observed the EMT marker SNAIL in the 3 kPa hydrogel but not in the 0.1 kPa hydrogel. Softer substrates are also more biologically relevant for a developing kidney: our organoids biologically resemble an embryonic kidney, which has a stiffness of <1 kPa, while our 3 kPa hydrogel is similar to the stiffness of an adult kidney of 5-10 kPa. These finding combined show increased evidence that a softer hydrogel correlates with better performance of organoids and reduced expression of EMT markers.

### Fast-Relaxing Dynamic Hydrogel Further Reduced the Undesired EMT-Related Phenotype

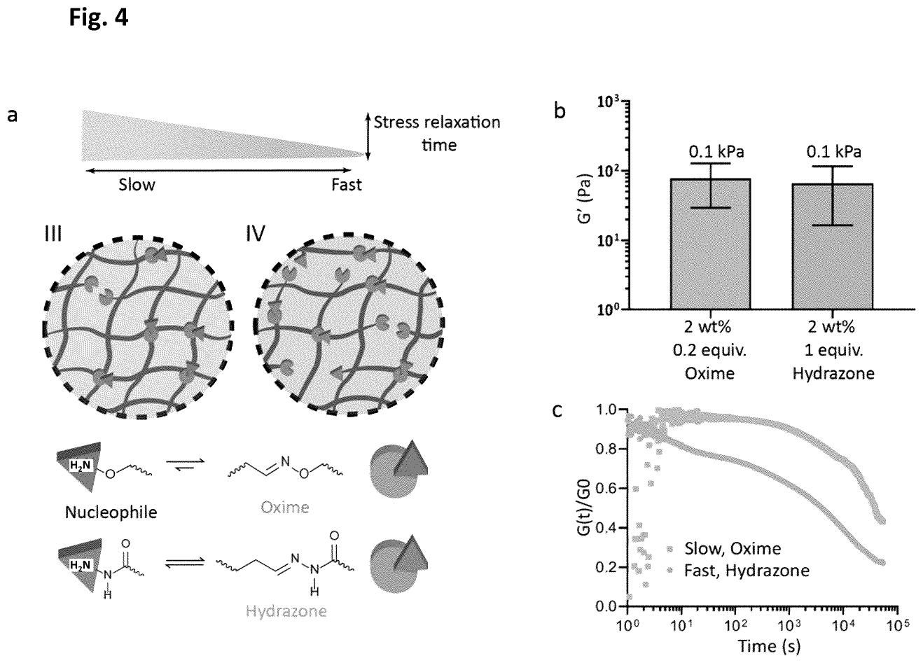

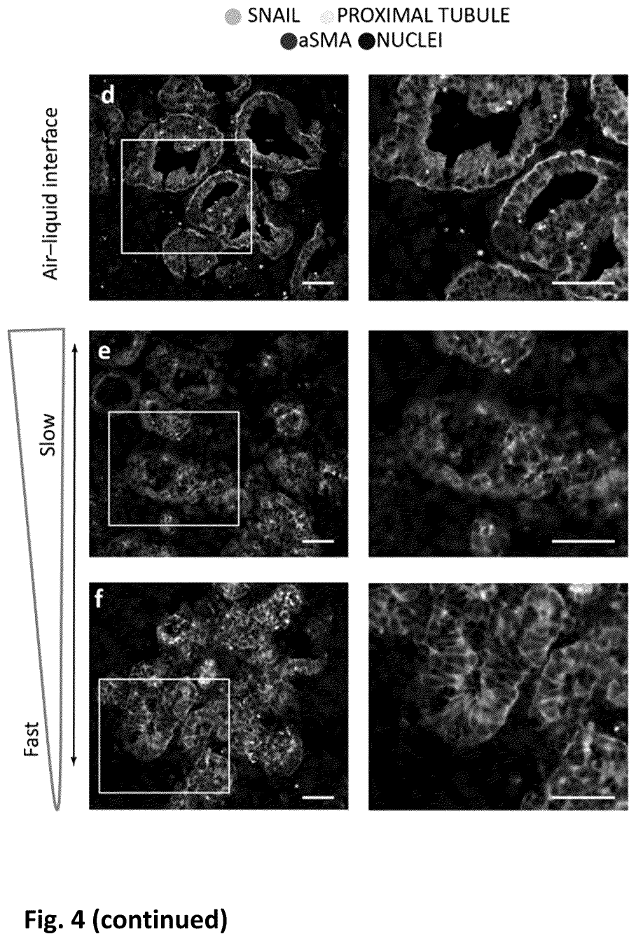

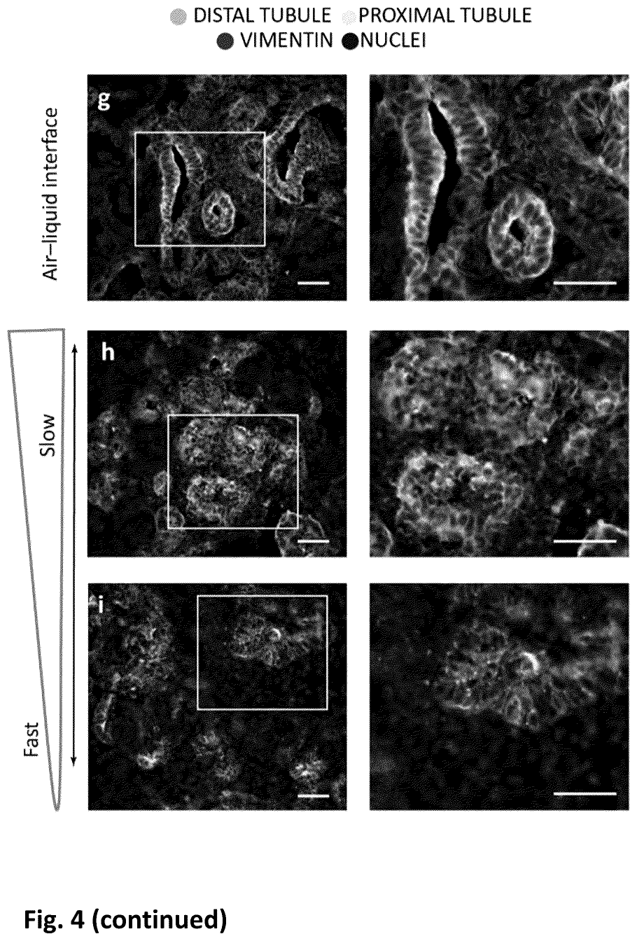

Beside stiffness (expressed as the shear moduli), stress relaxation plays a role in the cellular response to its surrounding material. We had so far kept the stress relaxation of the hydrogel similar (FIG. 1d) to compare only the effect of stiffness (FIG. 1c). To examine the effect of stress relaxation, we designed a fourth hydrogel with a similar stiffness as the soft oxime cross-linked hydrogel (˜0.1 kPa, FIG. 4a-b) but with a faster stress relaxation time by using hydrazone as the cross-linker (FIG. 4a). The imine group hydrolysis rate (k₋₁) can be tuned by changing the electronegativity of the alpha group to the primary amine. In this case, we decreased the electronegative alpha group by using hydrazone (adipic dihydrazide) instead of oxime to form a faster stress-relaxing hydrogel (FIG. 4a). A higher concentration of the cross-linker hydrazone (10.1 μM) with 2% oxidised alginate was used to obtain a hydrogel of similar stiffness as the soft oxime hydrogel of 0.1 kPa (FIGS. 4b and S10a-b). The characteristic stress relaxation time of the resulting hydrazone hydrogel is an order of magnitude faster, with a t_{1/2} value of 4.1×10³ s, compared to the 3.9×10⁴ s for the soft 0.1 kPa oxime hydrogel (FIG. 4c).

Organoids were encapsulated in the soft and faster stress-relaxing hydrogel at day 7+14 of culture, and were cultured to day 7+18. We detected no difference in the morphology of the organoids (bright-field imaging) or ratio of live/dead cells compared to organoids cultured on the air-liquid interface. Recovered organoids showed all nephron segments similar to organoids cultured on the air-liquid interface. A higher decrease of type 1a1 collagen expression was observed in organoids cultured in the soft, fast-relaxing hydrazone hydrogel compared to those cultured in the soft, slow-relaxing oxime hydrogel, while no change in type 6a1 collagen staining was observed compared to culture on the air-liquid interface. Interestingly, there was less aSMA, indicating presents of myofibroblasts, in organoids encapsulated in the fast-relaxing hydrazone hydrogels (FIG. 4f) compared to both those in the slow-relaxing, oxime cross-linked hydrogel (FIG. 4e) and on air-liquid interface (FIG. 4d), with no EMT-related SNAIL marker present in the fast-relaxing hydrazone hydrogel (FIG. 4f). Moreover, less expression of the mesenchymal marker vimentin was observed in the organoids encapsulated in the fast-relaxing hydrogel (FIG. 4i) compared to the slow stress-relaxing (FIG. 4h), and air-liquid interface (FIG. 4g).

### Soft Fast-Relaxing Hydrogels Result in Apical LTL Enrichment and Increased Ciliary Length and Frequency

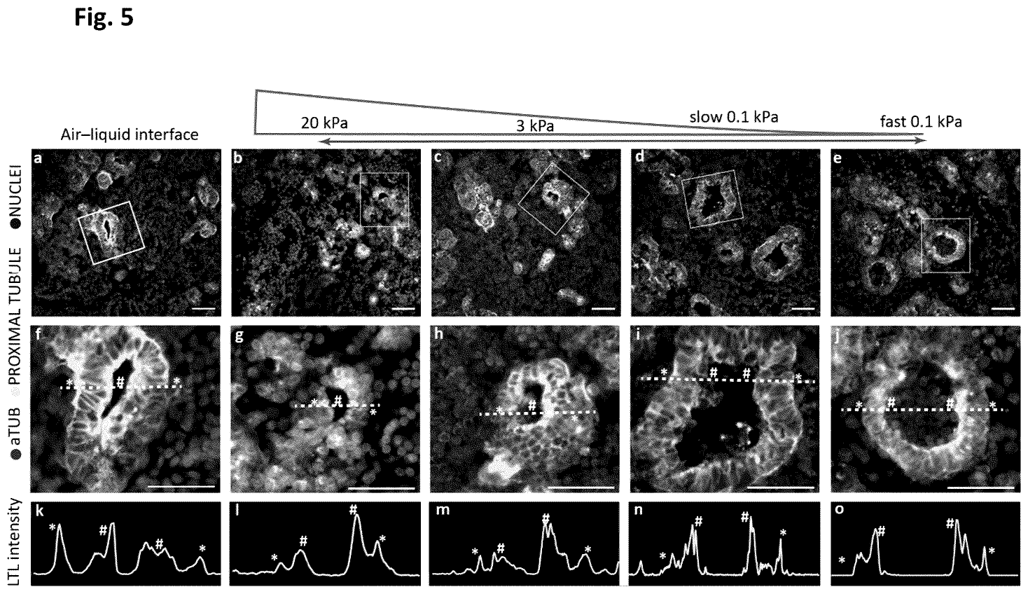

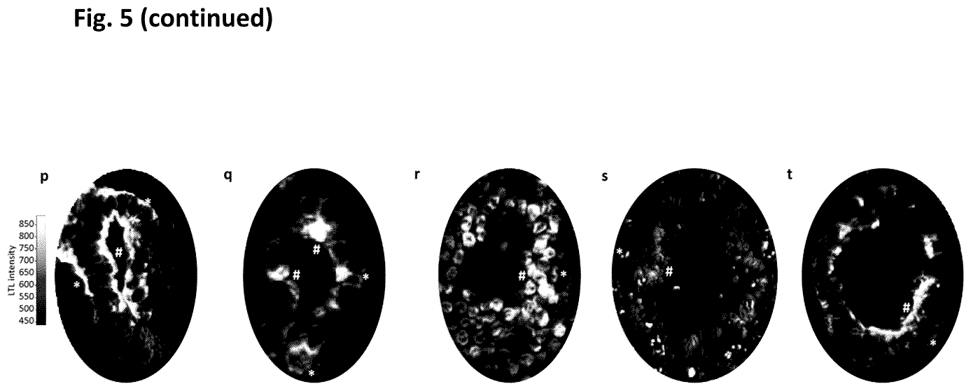

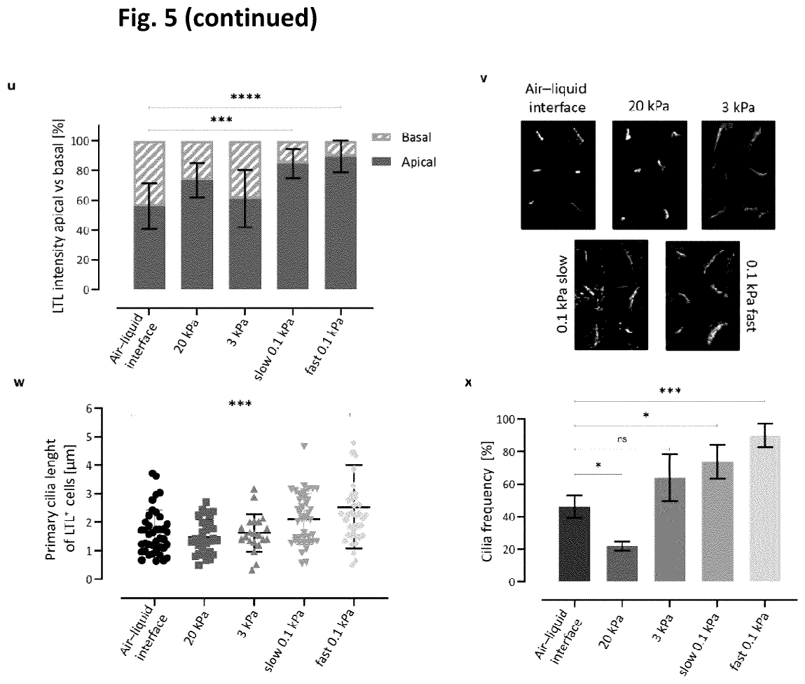

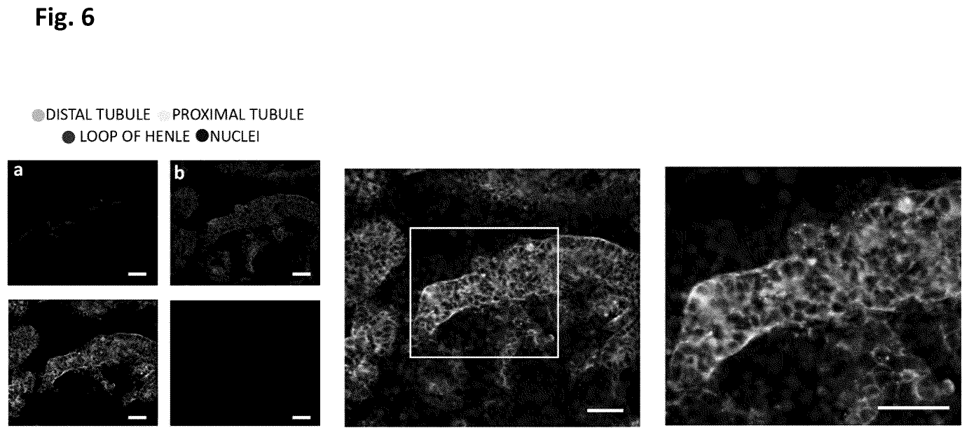

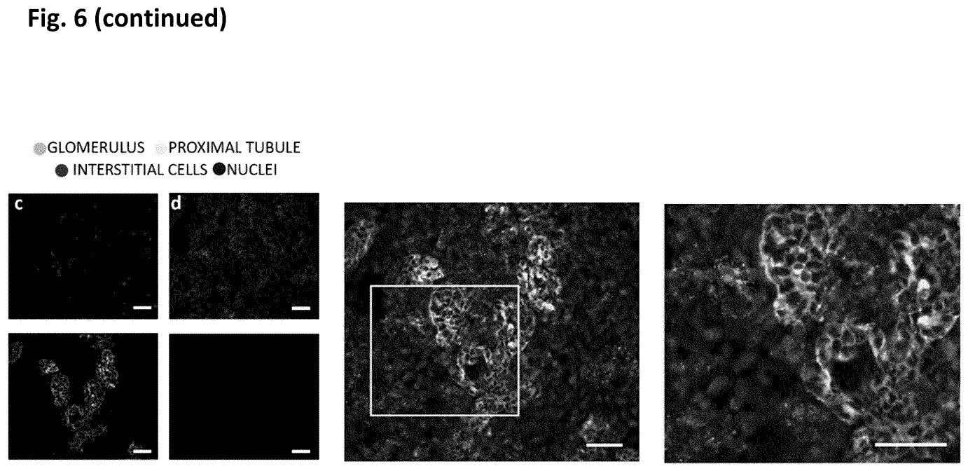

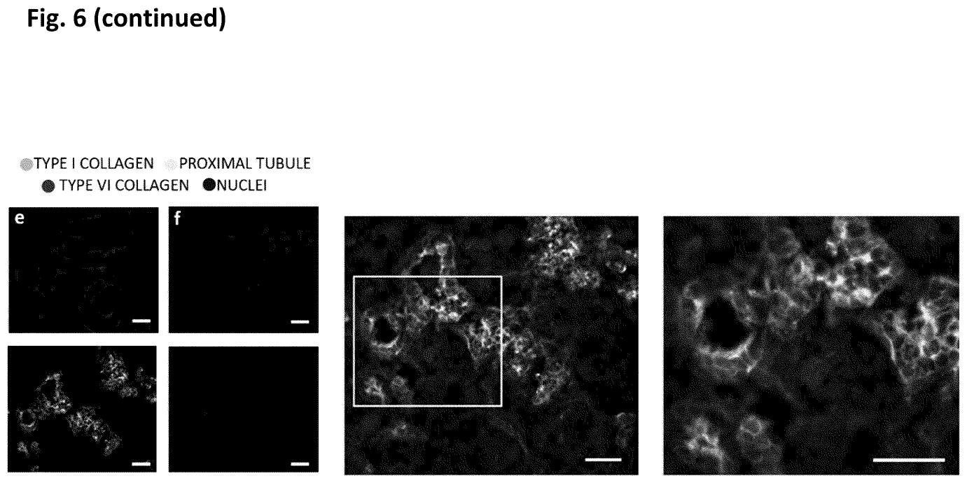

Differences in lumen structure of the organoids cultured on the different hydrogels were observed, which we could attribute to the stiffness and stress relaxation characteristics of the hydrogels. A significant increase of apical enrichment was observed in the LTL⁺ lumen of the organoids cultured in the two soft hydrogels (FIG. 5f-k-p-u-v and S9g-j), while no clear lumina were observed in the stiffest hydrogels (20 kPa, FIG. 5b-h-m-r and S9c-d). This indicated a link between stiffness and stress-relaxation on the lumen maturation, in which the softer fast-relaxing hydrogels increased the maturity.

We hypothesised a link between the observed lumen structures and the accumulated stress the organoids experience through confinement in the stiffer hydrogels. Primary cilia play an essential role in sensing environmental cues (e.g. mechanotransduction), planar cell polarity of epithelial cells, lumen formation, and EMT/fibrosis after acute kidney injury, in which many factors (such as, chemical or physical) can modulate the ciliary length and frequency. Therefore, we stained for primary cilia (acetylated a-tubulin (aTUB)) to investigate whether ciliary frequency and length vary in the different hydrogels. High-resolution z-stack confocal images showed differences in ciliary length (FIGS. 5v and S13). Significantly increased average ciliary length was observed in the softer hydrogels, 2.3 (p=0.0002) and 2.7 μm (p<0.0001) for slow and fast-relaxing 0.1 kPa, respectively, compared to 1.6 μm for the air-liquid interface (all cell types). During kidney development, ciliary length in the nephron lumen significantly increases from 0.59 to 3.04 μm from renal vesicles to fetal nephron, respectively. When only comparing proximal tubular ciliary length, we observed a significant increase in length of 2.5 μm (p=0.0002) in the soft fast-relaxing hydrogel compared to 1.6 μm for the air-liquid interface (FIG. 5w).

EMT can also trigger ciliary frequency deficiency and reduced length. The stiffest hydrogel (20 kPa) resulted in a significant deficiency of ciliary frequency (% of cilia containing cells, p=0.029, FIG. 5x), while 3 kPa does not show a significant difference. Both these hydrogels show EMT-related markers. In contrast, a significant increase in ciliary frequency was observed in the soft slow and fast relaxing hydrogels (p=0.015, p=0.0006, respectively, FIG. 5x). This clearly shows that the organoids feel the mechanic of the hydrogel confining environment, in which a higher stiffness (20 kPa) results in deficiency of cilia frequency and length, while softer hydrogels significantly increase the frequency (0.1 kPa). However, only the fast-relaxing hydrogel shows a significant increase in cilia length, as well as, frequency. This may be due to the fast stress-relaxing dynamics of the hydrogel network, which disperse tension through their hydrogel, reducing tension in the overall organoids. If we combine the significant increased ciliary length and frequency with the clear reduction of mesenchymal cells (VIM) and aSMA expression, and LTL apical enrichment in the organoids encapsulated in the soft fast-relaxing hydrogel, we can postulate a link between hydrogel stiffness and stress-relaxation on lumen maturation. The ability to disperse tension by hydrogel confinement in a fast stress-relaxing hydrogel results in a reduction of off-target cell types and increased lumen maturation.

### Methods

### Oxidised Alginate (Oxi-Alg) Synthesis

Sodium alginate was oxidised (10%) as previously described (Hafeez S, et al. Gels 2018, 4(4): 85). Briefly, purified sodium alginate (1.0 g, 1 equiv., 5.68×10⁻³ mol monomer, Manugel GMB, FMC, Lot No. G940200) was dissolved in 100 mL deionised H₂O overnight. Sodium (meta)periodate (0.121 g, 0.1 equiv., 5.68×10⁻⁴ mol, Sigma-Aldrich) was added. The mixture was covered with aluminium foil and stirred in the dark for 17 h at room temperature (RT). The reaction was quenched by the addition of ethylene glycol (0.035 g, 0.1 equiv. 5.68×10⁻⁴ mol, Sigma-Aldrich) and stirred for 1 h in the dark at RT. The resultant product was dialysed in a 10 kDa MWCO dialysis tube (Spectra/Por, regenerated cellulose, VWR) for 3 d in 100 mM, 50 mM, 25 mM, 12.5mM and 0 mM NaCl in deionised H₂O (changed twice daily), and was flash-frozen in liquid N₂ and lyophilised. Oxidation was confirmed by ¹H-NMR in deuterated water (D₂O) by the appearance of the protons between 5.15 and 5.75 ppm, attributed to the formation of hemiacetal groups upon reaction of the aldehydes to neighbouring hydroxyl groups. Moreover, molecular weights of the product were determined via GPC.

### ¹H-NMR Analysis

¹H-NMR spectra were recorded on a Bruker Avance III HD 700-MHz spectrometer equipped with a cryogenically cooled three-channel TCI probe in D₂O with sodium trimethylsilylpropanesulfonate as an internal standard (DSS, 2×10⁻³ M). Water suppression pulse sequence was applied to spectra. Spectra analyses were performed with MestReNova 11.0 software. Chemical shifts are reported in parts per million (ppm) relative to DSS (CH₂, 0 ppm).

### GPC Analysis

Agilent PEG calibration kit (PEG molecular weights up to 300,000 MW, Agilent Technologies) were used for calibration. The samples were dissolved at a concentration of 2 mg/mL in 0.1 M NaNO₃ H₂O and filtered through 0.45 μm filters to remove any unwanted impurities. Samples MW were measured in 0.1 M NaNO₃ H₂O eluent with a flow rate of 0.5 mL/min at RT on a Prominence-I LC-2030C3D LC (Shimadzu Europa GmbH) and Shodex SB-803/SB-804 HQ columns (Showax Denko America, Inc). LabSolutions GPC software (Shimadzu Europa GmbH) was used to calculate the molecular weight and dispersity values.

### Rheometry

Rheological characterisation on the hydrogels were performed on a DHR2 rheometer from TA Instruments. Time sweeps of preformed hydrogels were taken over 360 s with an 8 mm parallel plate geometry at 20° C. with an applied strain of 1% at 3.14 rad/s. During loading, the gap size was adjusted to achieve 0.1 N of normal force and varied between samples from 800 μm to 1150 μm with a mean of 1004±76 μm. The shear modulus (G′) for a given sample was taken to be the mean recorded value with a minimum of three sample replicates per formulation (FIGS. 1c and 5b). Stress relaxation measurements were performed using a 20 mm cone-plate geometry equipped with solvent trap. Precursor solutions were mixed as described in Table 3 to obtain the desired final concentrations of oxidised alginate and cross-linker. Following the final addition of the cross-linker, samples were vortexed for 10 s before immediately loading 80 μL into the rheometer. Time sweeps were measured over 3.5-9 h (maintained at 20° C.) to monitor crosslinking progress with an applied strain of 1% at 10 rad/s. Once a plateau was reached, a frequency sweep was performed from 100-0.1 rad/s with an applied strain of 1% and 10 pts/dec. Finally, to measure the stress relaxation behaviour, the relaxation modulus was monitored over 15.5 h with an initial applied strain of 20% maintained over the course of the measurement (FIGS. 1d and 5c). Statistical analysis were performed in GraphPad 8.2.0 using one-way ANOVA.

### Swelling Test of Hydrogels Under Culture Conditions

A swelling test to investigate real-world swelling under culture conditions was performed on transwell filters, as previously described (Geuens T, et al. Biomaterials 2021, 275, 120976. https://doi.org/10.1016/j.biomaterials.2021.120976). Briefly, hydrogel solutions were prepared (Table 2). 500 μL of this solution was added onto the transwells with a 0.4 μm pore size (Corning, 12-well culture plate) without organoids and left to cross-link for 1 h. After which, STEMdiff APEL2 medium (1% (v/v) PFHM-II protein-free hybridoma medium (Thermo Fisher Scientific) and 1% (v/v) antibiotic/antimycotic (Thermo Fisher Scientific) was added below the transwells with hydrogels and incubated up to 98 h (37° C.). Transwells with hydrogels were weighted at 1, 2, 4, 6, 24, 48, 72, and 96 h. Hydrogel swelling ratio in % (Sr) over time was calculated by:

$${S}_{r} = \frac{{w}_{x} - {w}_{o}}{{w}_{o}} ,$$

in which w₀=initial hydrogel weight and wₓ=hydrogel weight at the x time point. Graphad 8.2.0 software was used for statistical analysis using two-way ANOVA or unpaired t-test.

### Cell Culture

As described previously (Geuens T, et al. Biomaterials 2021, 275, 120976.https://doi.org/10.1016/j.biomaterials.2021.120976), hiPSC line LUMC0072iCTRL01 was generated from fibroblasts using the Simplicon RNA reprogramming kit (Millipore) by the hiPSC core facility at the Leiden University Medical Center. The cells were expanded in E8 medium (Thermo Fisher Scientific) on vitronectin-coated (0.5 μg cm⁻²) plates and passaged with TrypLE Express (Thermo Fisher Scientific) twice weekly.

For 24 h after each passage, cells were cultured in E8 medium supplemented with RevitaCell Supplement (Thermo Fisher Scientific). Subsequently, cells were cultured in E8 medium refreshed daily.

### Differentiation and Organoid Formation

Kidney organoids were produced from hiPSCs according to an established protocol (FIG. 1a) (van den Berg CW, et al. Stem Cell Reports 2018, 10(3): 751-765). Briefly, hiPSCs were seeded on 6-well plates with vitronectin-coating (0.5 μg cm⁻²), at 7,300 cells per cm² in E8 medium supplemented with RevitaCell Supplement. Differentiation was started after 24 h (day 0) by changing to STEMdiff APEL2 medium (STEMCELL Technologies) supplemented with CHIR99021 (8 μM, R&D Systems), 1% (v/v) antibiotic/antimycotic (Thermo Fisher Scientific), and 1% (v/v) PFHM-II protein-free hybridoma medium (Thermo Fisher Scientific). On day 4 of differentiation, medium was changed to STEMdiff APEL medium supplemented with heparin (1 μg ml⁻¹, Sigma-Aldrich) and FGF-9 (200 ng ml⁻¹, R&D Systems). By day 7, the cells formed a confluent monolayer and were subjected to a 1 h pulse of CHIR99021 (5 μM) in STEMdiff APEL medium before being harvested by trypsinisation. An aggregate of 500,000 cells was transferred to a Transwell filter with 0.4 μm pore size (Corning) in a 12-well culture plate to form the kidney organoids. Kidney organoids were cultured for 4 ds (termed 7+4) on an air-liquid interface with STEMdiff APEL2 medium supplemented with FGF-9 and heparin in the bottom compartment (450 μL) with media changes every 2 d. At day 7+5, STEMdiff APEL2 medium with no supplemented growth factors was used with media changes every 2 d. The organoids were maintained until day 7+18, with or without hydrogel encapsulation from day 7+14 (FIG. 1a-b).

### Hydrogel Encapsulation

Oxidised alginate (120 mg, 10% oxidation, 6 wt % stock solution, UV sterilised, Oxi-alg) was dissolved in 2 mL STEMdiff APEL2 medium and left to dissolve overnight. O,O′-1,3-propanediylbishydroxylamine dihydrochloride (oxime, Sigma-Aldrich) or adipic dihydrazide (hydrazone, Sigma-Aldrich) stock solutions of 8×10⁻² mol/mL were prepared in STEMdiff APEL2 medium (Table 3 and passed through a 0.2-micron sterilisation filter. The oxi-alg, crosslinker stock solutions, and STEMdiff APEL2 medium were added to obtain the desired hydrogel systems (Table 3) and vortexed before organoid encapsulation to form a 2% or 4% (w/v) sodium alginate solution. The hydrogel solutions were pipetted over the organoids on the top of the Transwell membrane at day 7+14 of culture (500 μL each). STEMdiff APEL2 medium (450 μL) was added to below the Transwell filters, and organoids encapsulated in the hydrogels were cultured for 4 additional days (until day 7+18, FIG. 1A-B).

### Cryo-Sectioning Recovered Organoids

At day 7+18, the hydrogels were removed and 4% paraformaldehyde (PFA) was added above and below the Transwell with the recovered organoid for 20 min at 4° C. Organoids were cryo-sectioned as described previously (Geuens T, et al. Biomaterials 2021:120976.). Briefly, organoids were dehydrated overnight (PBS containing 15% (w/v) sucrose) at 4° C. followed by a second 2-day dehydration incubation (30% (w/v) sucrose). The dehydrated organoids were embedded in freezing solution (15% (w/v) sucrose and 7.5% (w/v) gelatin in PBS). The embedded organoids were placed in a beaker with isopentane and left to freeze in liquid N₂ for several minutes. The frozen organoids were horizontally sectioned to 20 μm thickness at −18° C.

### Immunohistochemistry

The embedding solution of the frozen organoid sections was removed by incubating for 15-20 min in PBS at 37° C. The sections were washed (PBS), permeabilised (PBS with 0.5% (v/v) IGEPAL) for 15 min (RT), blocked (PBS with 5% (w/v) donkey serum, 1% BSA and 0.3M glycine) for 20 min (RT) and incubated overnight at 4° C. (in the dark) with primary antibodies (PBS with 1% BSA and 0.3M glycine) against: nephrin or NPHS1, lotus tetragonolobus lectin or LTL, Meis Homeobox 1/2/3 or MEIS1/2/3, E-cadherin or ECAD, solution carrier family 12 Member 1 or SLC12A1, type 1a1 and type 6a1 collagen, aSMA, vimentin, acetylated tubulin or a-tubulin, and Zinc finger protein SNAI1 (Table. 1). Subsequently, the slides were washed three times with PBS (1% BSA and 0.3M glycine) and incubated with secondary antibodies including DAPI (0.1 μg/ml) for 1 h at RT in the dark: Alexa Fluor 488 (Thermo Fisher Scientific, 1:300, sheep/mouse/rabbit), Alexa Fluor 568 (Thermo Fisher Scientific, 1:300, mouse/rabbit), and Streptavidin Alexa Fluor 647 (Thermo Fisher Scientific, 1:100). Slides were washes three time in PBS and mounted with Mowiol mounting medium. Images were taken with an automated Nikon Eclipse Ti2-E microscope (20× or 40× air objective) or light microscope Leica TCS SP8 STED (100× objective).

TABLE 1
Primary antibodies used in this study.

| | Mono- | Source | | | | |
|---|---|---|---|---|---|---|
| Primary | or | (catalog | | Membrane | | |
| Antigen | Polyclonal | no.) | Species | or nucleus | Identifies | Dilution |
| Acetylated | Mono | MERCK | Mouse | Membrane | Primary Cilia | (1:750) |
| α-Tubulin | | (T7451) | | | | |
| (aTUB) | | | | | | |
| aSMA | Mono | Sigma- | Mouse | Cytoplasm/ | Fibroblasts | (1:300) |
| | | Aldrich | | ECM | | |
| | | (A2547) | | | | |
| ECAD | Mono | BD | Mouse | Membrane | Distal tubule | (1:300) |
| | | Biosciences | | | | |
| | | (610181) | | | | |
| LTL | — | Vectorlabs | Biotin | Membrane | Proximal | (1:300) |
| | | (B-1325) | conjugate | | tubule | |
| MEIS1/2/3 | Mono | Santa- | Mouse | Nucleus | Interstitial | (1:300) |
| | | Cruz | | | cells | |
| | | Biotech- | | | | |
| | | nology (SC- | | | | |
| | | 101850) | | | | |
| NPHS1 | Poly | R&D | Sheep | Membrane | Podocytes | (1:300) |
| | | Systems | | | | |
| | | (AF426)9 | | | | |
| SLC12A1 | Poly | Thermo | Rabbit | Membrane | Loop of | (1:200) |
| | | Fisher | | | Henle | |
| | | Scientific | | | | |
| | | (HPA014967) | | | | |
| SNAIL | Mono | Thermo | Rabbit | Nucleus | EMT | (1:300) |
| | | Fisher | | | transcription | |
| | | Scientific | | | factor | |
| | | (PA5- | | | | |
| | | 85493) | | | | |
| Type I | Mono | Abcam | Mouse | ECM | Type 1 | (1:300) |
| Collagen | | (ab6308) | | | Collagen | |
| (COL1A1) | | | | | | |
| Type VI | Mono | Genetex | Rabbit | ECM | Type 6 | (1:300) |
| Collagen | | (GTX109963) | | | Collagen | |
| (COL6A1) | | | | | | |
| Vimentin | Mono | Thermo | Mouse | ECM | EMT ECM | (1:300) |
| (VIM) | | Fisher | | | marker | |
| | | Scientific | | | | |
| | | (MA3745) | | | | |

### Cell Viability Assay

EthD1/calcein AM staining were used to determine cell viability. The encapsulating hydrogel and medium were removed. Recovered organoids on Transwells were incubated in EthD1 (4 μM) and calcein AM (2 μM) in PBS solution (top and bottom of the Transwell) for 30 min at RT. Organoids were imaged in PBS with an automated Nikon Eclipse Ti2-E microscope at 4×, 20×, or 40× air objective.

### Analysis of EMT-Related Gene Expression in Kidney Organoids

Single-cell RNAseq data of iPSC-derived kidney organoids generated using the Takasato protocol (Wu H, et al., Cell Stem Cell 2018, 23(6): 869-881) were downloaded from the Gene Expression Omnibus (GEO: GSE118184) and analysed as previously described (Geuens T, et al. Biomaterials 2021: 120976). Briefly, transcript count tables were analysed for each time point (day 0, 7, 7+5, 7+12, 7+19 and 7+27) by R software (3.6.2) and the Seurat package (version 3.2.0);with the exclusion of low-quality cells. The gene expression matrices were log-transformed using a scaling factor of 10,000 and normalised for sequencing depth per cell. Subsequently, the highest cell-to-cell variations were identified, scaled, and centred. These data were used for principal component analysis. Non-linear dimensional reduction was performed on selected principal components representing the true dimensionality of each dataset. Normalised markers gene expression of interest are presented in tSNE space.

### LTL Polarity of Lumen Structures

Immunohistochemistry images were processed in ImageJ. Plot profiles were analysed in ImageJ and heatmaps were generated by the interactive 3D surface plot plugin. Percentage of basal vs apical intensity were calculated by deducting plot profile from apical side from the full plot profile per lumen structure. (N=3 organoid batches, n=9 images per condition were processed). Statistical analysis were performed in GraphPad 8.2.0 using two-way ANOVA.

### Primary Cilia Length and Frequency Measurements

Z-stack confocal images measured (100× objective) were processed in ImageJ to Z projection. Length per cilia were measured using the straight-line function (N=3 organoid batches, n=5 images per condition were processed). Cilia frequency were measured using the cell counter plugin, cilia frequency were calculated by measuring the percentage of nuclei with primary cilia compared to the overall nuclei (N=3 organoid batches, n=3 images per condition were processed). Statistical analysis were performed in GraphPad 8.2.0 using two-way ANOVA.

TABLE 2
Molecular weight averages by GPC. Number average (Mₙ), weight
average (M_{w}), and dispersity (M_{w}/Mₙ) for pure alginate and two
oxidised alginate (Oxi-Alg) batches used for the study.

| | Mₙ [kDa] | M_{w} [kDa] | M_{w}/Mₙ [D] |
|---|---|---|---|
| Pure Alg | 130.7 | 316.2 | 2.46 |
| Oxi-Alg1 | 28.3 | 69.6 | 2.46 |
| Oxi-Alg2 | 83.5 | 185.3 | 2.21 |

TABLE 3
Volume, weights, and other properties of the prepared stock solutions
and hydrogel solutions to form four different cross-linked
alginate hydrogel systems used in these studies.
Stock solutions

| | | STEMdiff | | |
|---|---|---|---|---|
| | Weight | APEL2 or PBS | [mol/ | |
| Chemical | [mg] | [mL] | mL] | Wt % |
| Oxi-alg-1 | 165 | 2.75 | — | 6 |
| or -2 | | | | |
| Oxime | 15 | 1.05 | 8.00 × | — |
| | | | 10⁻² | |
| Hydra- | 15 | 1.08 | 8.00 × | — |
| zone | | | 10⁻² | |

Hydrogel solutions and properties for rheometry

| | Oxi- | | | | |
|---|---|---|---|---|---|
| | alg (-1 | | | Cross- | |
| | or -2) | Crosslinker | | linker | Oxi-alg [% |
| Hydrogel | [μL] | [μL] | PBS [μL] | [μM] | w] |
| 0.1 kPa | 51.7 | Oxime, | 99.4 | 2.02 | 2 |
| | (1) | 3.9 | | | |
| 3 kPa | 51.7 | Oxime, | 83.7 | 10.1 | 2 |
| | (1) | 19.6 | | | |
| 20 kPa | 103.3 | Oxime, | 12.5 | 20.2 | 4 |
| | (1) | 39.2 | | | |
| Fast 0.1 | 51.7 | Hydrazone, | 83.7 | 10.1 | 2 |
| kPa | (2) | 19.6 | | | |

Hydrogel solutions and properties for organoid
culture and swelling test

| | Oxi- | | STEMdiff | | Oxi- | |
|---|---|---|---|---|---|---|
| | alg (-1 | | APEL2 | Cross- | alg | Stiff- |
| Hydrogel | or -2) | Crosslinker | medium | linker | [% | ness |
| code | [μL] | [μL] | [μL] | [μM] | w] | [kPa] |
| 0.1 kPa | 166.7 | Oxime, | 320.7 | 2.02 | 2 | 0.08 |
| | (1) | 12.6 | | | | |
| 3 kPa | 166.7 | Oxime, | 270.2 | 10.1 | 2 | 3 |
| | (1) | 63.2 | | | | |
| 20 kPa | 333.3 | Oxime, | 40.4 | 20.2 | 4 | 20 |
| | (1) | 126.3 | | | | |
| Fast 0.1 | 166.7 | Hydrazone, | 320.7 | 10.1 | 2 | 0.1 |
| kPa | (2) | 63.2 | | | | |

1. A method of generating a hydrogel for organoid culture, the method comprising:
providing a suspension of a polymer;
obtaining polymer with aldehyde groups; and
cross-linking the polymer with aldehyde groups with a cross-linking agent to obtain a cross-linked polymer; and
allowing the suspension comprising the cross-linked to form a hydrogel,
wherein the amount of oxidizing agent, the amount of cross-linking agent and amount of polymer are chosen such that the resulting hydrogel has a stiffness between 0.01 and 4 kPa as measured by shear moduli on a DHR2 rheometer from TA Instruments, and
wherein the hydrogel has a stress relaxation time (t1/2) of 104 seconds or less as measured by relaxation modulus on a DHR2 rheometer from TA Instruments.

2. The method according to claim 1, wherein the polymer is sodium alginate, hyaluronic acid, PEG, PEG derivatives, gelatin, GelMA, dextran, PVA, PAA, PAcm, collagen, or peptide modified polymers thereof, or mixtures thereof.

3. The method according to claim 1, wherein the stiffness is between 0.01 and 1 kPa, more preferably between 0.01 and 0.5 kPa, as measured by shear moduli on a DHR2 rheometer from TA Instruments.

4. The method according to claim 1, wherein the oxidizing agent is an oxygen-atom transfer (OAT) agent, preferably wherein the oxygen-atom transfer (OAT) agent selected from a periodate salt such as NaIO4, a permanganate salt such as KMnO4, H2O2, a chromate salt, OsO4, a perchlorate salt or combinations thereof.

5. The method according to claim 1, wherein the cross-linking agent is a compound having at least two amine groups, preferably wherein the cross-linking agent is selected from the group consisting of
an alkoxy compound having formula (I): 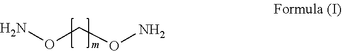
wherein m is 2 to 12,
a semicarbazide compound having formula (II): 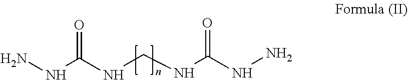
wherein n is 2 to 12, and
a hydrazide compound having formula (III): 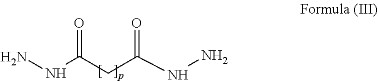
wherein p is 2 to 12
wherein 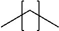
represents an alkylene group having m, n or p carbon atoms, wherein 1 or more carbon atoms can be replaced by a heteroatom selected from O, S and N;
a compound having formula (IV): 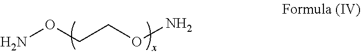
wherein x is 1 to 250;
a compound having formula (V): 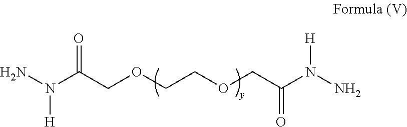
wherein y is 1 to 250;
a compound having formula (VI): 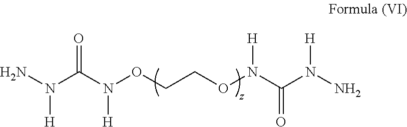
wherein z is 1 to 250;
a compound having formula (VII) 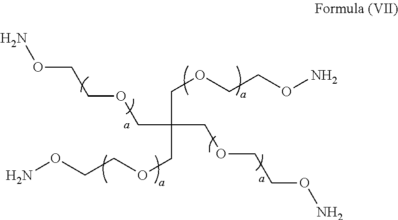
wherein each a is individually 1 to 250;
a compound having formula (VIII) 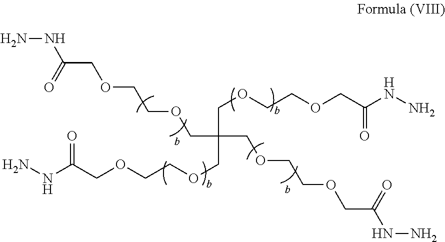
wherein each b is individually 1 to 250;
a compound having formula (IX): 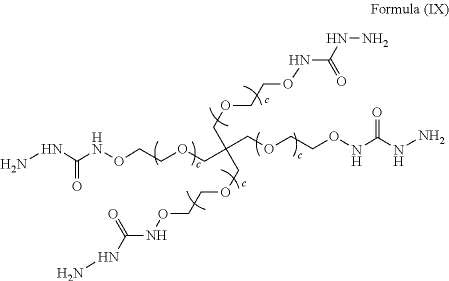
wherein each c is individually 1 to 250;
a compound having formula (X): 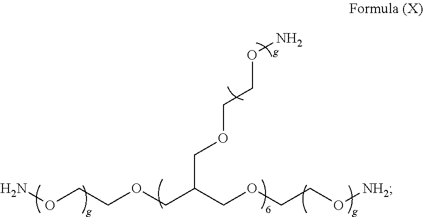
wherein each g is individually 1 to 250
a compound having formula (XI) 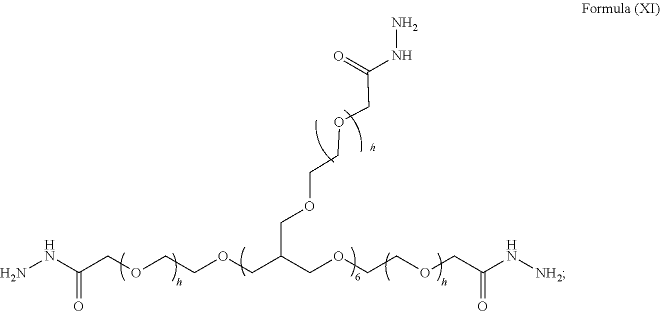
wherein each h is individually 1 to 250
a compound having formula (XII): 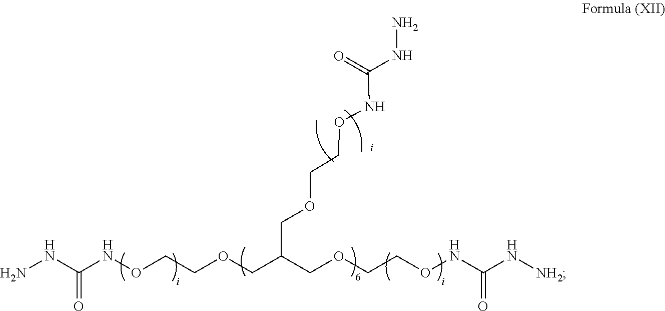
wherein each i is individually 1 to 250
a compound having formula (XIII): 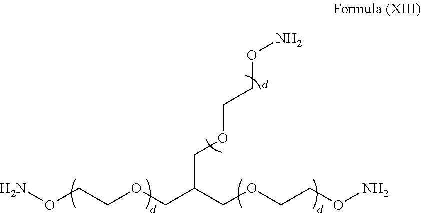
wherein each d is individually 1 to 250;
a compound having formula (XIV) 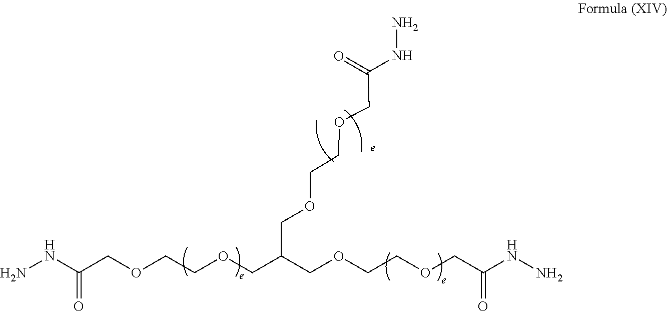
wherein each e is individually 1 to 250;
a compound having formula (XV): 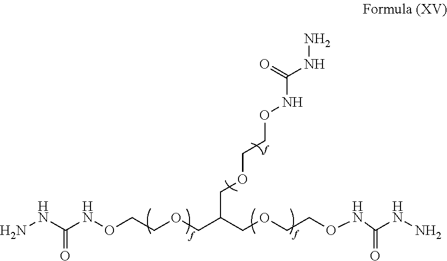
wherein each f is individually 1 to 250;
a compound having formula (XVI): 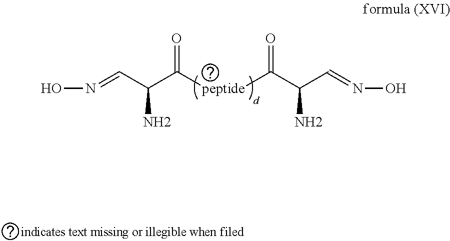
wherein 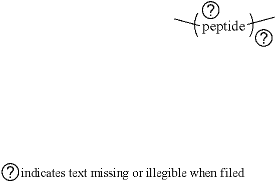
represents a peptide of 2 to 45 amino acids, preferably an enzymatically cleavable peptide; and
a compound having formula (XVII): 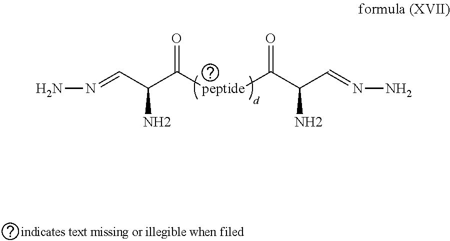
wherein 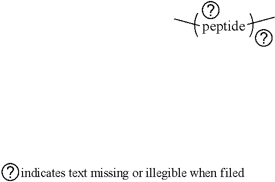
represents a peptide of 2 to 45 amino acids, preferably an enzymatically cleavable peptide;
preferably wherein the crosslinker is O,O′-1,3-propanediylbishydroxylamine or adipic dihydrazide.

6. The method according to claim 1, wherein the hydrogel is further used to culture an organoid or cell aggregate, the method further comprising the steps of:
culturing a cell, preferably a stem cell, induced pluripotent stem cell (iPSC), adult stem cell, embryonic stem cell, or progenitor cell under conditions to allow an organoid or cell aggregate to form,
embedding the organoid or cell aggregate in or on the surface of the polymer suspension; and
culturing the organoid or cell aggregate in the hydrogel,
wherein the cross-linking step is performed on the oxidized polymer prior, during or after embedding the organoid or cell aggregate.

7. The method according to claim 1, wherein the organoid or cell aggregate is a kidney organoid, an intestinal organoid, a pancreatic organoid, a neural organoid, a hepatic organoid, a thyroid organoid, a stomach organoid, an ovarian organoid, a prostate organoid, a splenic organoid, oesophageal organoid, a breast organoid, a bladder organoid, a lung organoid, an optic organoid, an inner ear organoid, a cardiac organoid, a biliary organoid, a salivary gland organoid, a pituitary gland organoid, a lymphoid organoid, a bladder organoid, a tongue organoid, a cerebral/brain organoid, a spinal cord organoid, a fallopian tube organoid, a lacrimal gland organoid, a skin organoid, and a hippocampal organoid, or wherein the cell aggregate are mesenchymal stem cells or cells or progenitor derived thereof such a osteoblasts, osteoclasts, chondrocytes or adipocytes.

8. A hydrogel obtained or obtainable by the method according to claim 1.

9. The hydrogel according to claim 8 further comprising an organoid, a cell aggregate or cells, preferably wherein the organoid or cell aggregate is a kidney organoid, an intestinal organoid, a pancreatic organoid, a neural organoid, a hepatic organoid, a thyroid organoid, a stomach organoid, an ovarian organoid, a prostate organoid, a splenic organoid, oesophageal organoid, a breast organoid, a bladder organoid, a lung organoid, an optic organoid, an inner ear organoid, a cardiac organoid, a biliary organoid, a salivary gland organoid, a pituitary gland organoid, a lymphoid organoid, a bladder organoid, a tongue organoid, a cerebral/brain organoid, a spinal cord organoid, a fallopian tube organoid, a lacrimal gland organoid, a skin organoid, and a hippocampal organoid, or wherein the cell aggregate are mesenchymal stem cells or cells or progenitor derived thereof such a osteoblasts, osteoclasts, chondrocytes or adipocytes.

10. The hydrogel according to claim 9 wherein the organoid is a kidney organoid.

11. The hydrogel comprising an organoid or cell aggregate according to claim 9 for use as a medicament.

12. The hydrogel comprising an organoid or cell aggregate according to claim 9 for use in a method of preventing, treating or ameliorating a disease, the use comprising transplanting the hydrogel comprising the organoid or cell aggregate in a subject in need thereof.

13. The hydrogel comprising a kidney organoid according to claim 10 for use in a method of preventing, treating or ameliorating a kidney disease.

14. Use of the hydrogel according to claim 8 in one or more of:
drug screening,
drug testing,
drug discovery,
drug development,
drug validation,
culturing cells, cell aggregates or organoids,
scale up for organoids,
disease modelling,
organoid implementation,
as a model organ.

15. A method for culturing an organoid, the method comprising the steps of:
culturing a stem cell, induced pluripotent stem cell (iPSC) or progenitor cell under conditions to allow an organoid to form,
embedding the organoid in a hydrogel obtained or obtainable by the methods according to claim 1, and
culturing the organoid in the hydrogel,
wherein the cross-linking step of the hydrogel is performed prior, during or after embedding the organoid in the hydrogel.
